(19) United States
(12) Patent Application Publication
(10) Pub. No.: US 2024/0335614 A1
(43) Pub. Date: Oct. 10, 2024

(54) SERIAL DOSING DEVICE AND METHOD

(71) Applicant: CREDENCE MEDSYSTEMS, INC., Menlo Park, CA (US)

(72) Inventors: John Robert Lund, Los Altos, CA (US); Alan E. Shluzas, San Carlos, CA (US); Conor Edward Shanley, Emerald Hills, CA (US); Jeff Tillack, Wake Forest, NC (US)

(73) Assignee: CREDENCE MEDSYSTEMS, INC., Menlo Park, CA (US)

(21) Appl. No.: 18/628,606

(22) Filed: Apr. 5, 2024

Related U.S. Application Data
(60) Provisional application No. 63/494,741, filed on Apr. 6, 2023.

(51) Int. Cl. A61M 5/315; A61M 5/31; A61M 5/32

(52) U.S. Cl. CPC A61M 5/31505; A61M 5/3137; A61M 5/3245; A61M 2005/3254

(57) ABSTRACT

A system for medication dispensing includes a syringe body having proximal and distal ends, a syringe interior, a syringe flange at the proximal end thereof, and a drug disposed in the syringe interior. The system also includes a finger flange coupled to the syringe flange and defining a finger flange interior and a side window therethrough. The system further includes a stopper member disposed in the syringe interior. Moreover, the system includes a plunger member coupled to the stopper member and disposed at least partially within the finger flange. In addition, the system includes a thumbpad coupled to a proximal end of finger flange and operatively coupled to the plunger member. The system also includes a cam configured to rotate a predetermined angular distance and a ratchet configured to advance the plunger member a predetermined longitudinal distance with each full advancement of the thumbpad into the finger flange.

The present application claims priority to U.S. Provisional Patent Application Ser. No. 63/494,741, filed on Apr. 6, 2023 under attorney docket number CM.30042.00 and entitled “SERIAL DOSING DEVICE AND METHOD.” This application also includes subject matter similar to the subject matter described in the following co-owned U.S. patent applications: (1) Ser. No. 14/321,706, filed Jul. 1, 2014 under attorney docket number CM.20001.00, and entitled “SAFETY SYRINGE”, (2) Ser. No. 14/543,787, filed Nov. 17, 2014 under attorney docket number CM.20002.00, and entitled “SYSTEM AND METHOD FOR DRUG DELIVERY WITH A SAFETY SYRINGE”; (3) Ser. No. 14/696,342, filed Apr. 24, 2015 under attorney docket number CM.20003.00, and entitled “SYSTEM AND METHOD FOR SAFETY SYRINGE”; (4) Ser. No. 15/801,239, filed Nov. 1, 2017 under attorney docket number CM.20011.00, and entitled “SYSTEM AND METHOD FOR SAFETY SYRINGE”; (5) Ser. No. 15/801,259, filed Nov. 1, 2017 under attorney docket number CM.20012.00, and entitled “SYSTEM AND METHOD FOR SAFETY SYRINGE”; (6) Ser. No. 15/801,281, filed Nov. 1, 2017 under attorney docket number CM.20013.00, and entitled “CARTRIDGE SAFETY INJECTION SYSTEM AND METHODS”; (7) Ser. No. 16/011,453, filed Jun. 18, 2018 under attorney docket number CM.20014.00, and entitled “SYSTEM AND METHOD FOR SAFETY SYRINGE”; (8) Ser. No. 15/801,304, filed Nov. 1, 2017 under attorney docket number CM.20015.00, and entitled “SYSTEM AND METHOD FOR SAFETY SYRINGE”; (9) Ser. No. 15/985,354, filed May 21, 2018 under attorney docket number CM.20016.00, and entitled “SYSTEM AND METHOD FOR COLLECTING INJECTION INFORMATION”; (10) Ser. No. 16/683,157, filed Nov. 13, 2019 under attorney docket number CM.20022-2.00, and entitled “SYSTEM AND METHOD FOR MULTIPLE SITE INJECTION”; and (11) Ser. No. 63/390,049, filed Jul. 18, 2022 under attorney docket number CM.30038.00, and entitled “SAFE INJECTION SYSTEMS AND METHODS”. The contents of the above-mentioned applications are fully incorporated herein by reference as though set forth in full.

### FIELD OF THE DISCLOSURE

The present disclosure relates generally to medication dosing systems, devices, and processes for facilitating various levels of control over fluid or gel dispensing, infusion, and/or injection, and more particularly to systems and methods related to dosing systems for serial delivery of multiple doses of medication.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a proximal/retracted position relative to the syringe body (4). The tubular needle shield (22) is “locked” in the distal/extended configuration, such that the needle shield (22) can no longer be returned to the proximal/retracted position, to prevent accidental needle sticks after injection.

Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap (35) in place; FIG. 5B has the cap removed to illustrate the Luer interface 14). Liquid medicine may reside in the volume, or medicine reservoir, (40) between the distal seal and the distal end (37) of the plunger tip or stopper (36). The plunger tip or stopper (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus, certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Some medications are serially delivered to multiple sites in or on a patient during treatment. There is a need for dosing systems which address shortcomings of currently-available configurations. In particular, there is a need for dosing systems that serially dose fluids or gels for the treatment of one patient. It is also desirable that such syringe assemblies may utilize the existing and relatively well-controlled supply chain of conventionally delivered pre-filled syringes, cartridges and other off-the-shelf components, and the corresponding assembly machinery and personnel.

### SUMMARY

Embodiments are directed to injection systems. In particular, the embodiments are directed to injection systems for serial delivery of multiple doses of injectables.

In one embodiment, a system for medication dispensing includes a syringe body having proximal and distal ends, a syringe interior, and a syringe flange at the proximal end thereof. The system also includes a drug disposed in the syringe interior. The system further includes a finger flange coupled to the syringe flange. Moreover, the system includes a stopper member disposed in the syringe interior. In addition, the system includes a plunger member coupled to the stopper member and disposed at least partially within the finger flange. The system also includes a thumbpad coupled to a proximal end of finger flange and operatively coupled to the plunger member. The finger flange includes a finger flange body defining a finger flange interior and a side window therethrough. The finger flange also includes a ratchet configured to advance the plunger member a predetermined longitudinal distance with each full advancement of the thumbpad into the finger flange. The finger flange further includes a cam configured to rotate a predetermined angular distance with each full advancement of the thumbpad into the finger flange.

In one or more embodiments, the finger flange further includes a stator operatively coupled to the thumbpad and the cam, and configured to rotate the cam with advancement of the thumbpad into the finger flange. The cam may include at least one rotation indicia visible through the window in the finger flange body, and configured to indicate a number of full advancement of the thumbpad into the finger flange. The system may also include a spring configured to return the thumbpad proximally after distal depression of the thumbpad into the finger flange.

In one or more embodiments, the finger flange is configured to emit an audible indicator for each full depression of the thumbpad into the finger flange. The finger flange may be configured to prevent depression of the thumbpad into the finger flange after a predetermined number of full depressions of the thumbpad into the finger flange. The system may also include a semi-rigid distal cap including a shield body; and an elastic member disposed in the shield body.

The aforementioned and other embodiments of the invention are described in the Detailed Description which follows.

### BRIEF DESCRIPTION OF THE DRAWINGS

This patent or application file contains at least one drawing executed in color. Copies of this patent or patent application publication with color drawings will be provided by the U.S. Patent and Trademark Office upon request and payment of the necessary fee.

FIGS. 1A to 5C illustrate various aspects of conventional injection syringe configurations.

FIG. 6 is a longitudinal cross-sectional view of a multiple site dosing system according to some embodiments.

FIG. 7 is a detailed longitudinal cross-sectional view of a multiple site dosing system depicted in FIG. 6.

FIG. 8 is a detailed longitudinal cross-sectional view, taken along a plane orthogonal to the cross-section plane in FIG. 7, of the multiple site dosing system depicted in FIGS. 6 and 7.

FIG. 9 through 12 are longitudinal cross-sectional view of a multiple site dosing system schematically depicting a sequence of motions that occur within the device upon full advancement of the thumbpad according to some embodiments.

FIG. 13 is a perspective view of a multiple site dosing system according to some embodiments.

FIGS. 14A and 14B are side views of a multiple site dosing system including a removable cap according to some embodiments.

FIGS. 15A and 15B are side and longitudinal cross-sectional views of a removable cap according to some embodiments.

FIG. 16 is a side view of a serial dosing system according to some embodiments.

FIG. 17 is a partially exploded view of various components from the serial dosing system depicted in FIG. 16 according to some embodiments.

FIG. 18 is a longitudinal cross-sectional view of the serial dosing system depicted in FIG. 16.

FIG. 19A is a front perspective view of the serial dosing system depicted in FIG. 16 with the cap removed according to some embodiments.

FIG. 19B schematically depicts the distal end of the serial dosing system depicted in FIG. 16 with the cap removed dispensing a fluid or gel onto a finger of a user according to some embodiments.

FIGS. 20 to 22 are a detailed longitudinal cross-sectional view of the serial dosing system depicted in FIG. 16 during the delivery of a single dose of fluid or gel.

FIG. 23 is a side view of a thumbpad shaft of the serial dosing system of FIG. 16.

FIGS. 24A and 24B are proximal and distal perspective views of a cam ring of the serial dosing system of FIG. 16.

FIGS. 25 to 27 are distal (FIG. 25) and proximal (FIGS. 26 and 27) perspective views of a finger flange housing of the serial dosing system of FIG. 16.

FIGS. 28A and 28B are distal perspective views of distal and proximal portions of a ratchet of the serial dosing system of FIG. 16.

FIG. 29 is a distal perspective view of a ratchet for use with serial dosing systems according to some embodiments.

FIG. 30 is an exploded view of the finger flange of the serial dosing system of FIG. 16 and the components contained therein according to some embodiments.

FIGS. 31 to 35 are side perspective views schematically depicting a sequence of the dose counting mechanism of the serial dosing system of FIG. 16.

FIGS. 36 and 37 schematically depict indicia disposed on dose counting cams of serial dosing systems according to some embodiments, including the serial dosing systems described herein.

FIGS. 38 to 40 are side views depicting a serial dosing system with indicia indicating various dose counts according to some embodiments.

FIGS. 41 and 42 are longitudinal cross-sectional views along orthogonal axes of a serial dosing system according to some embodiments.

FIGS. 43 and 44 our detailed longitudinal cross-sectional views depicting the delivery of a dose using a serial dosing system according to some embodiments.

FIG. 45 is a distal perspective view of a dose counting cam for use with serial dosing systems according to some embodiments.

FIG. 46 is a detailed longitudinal cross-sectional view depicting a distal end of a serial dosing system with a removable cap attached according to some embodiments.

FIG. 47 is an exploded view of the removable cap picked it in FIG. 46.

FIG. 48 is a side view of a serial injection system according to some embodiments.

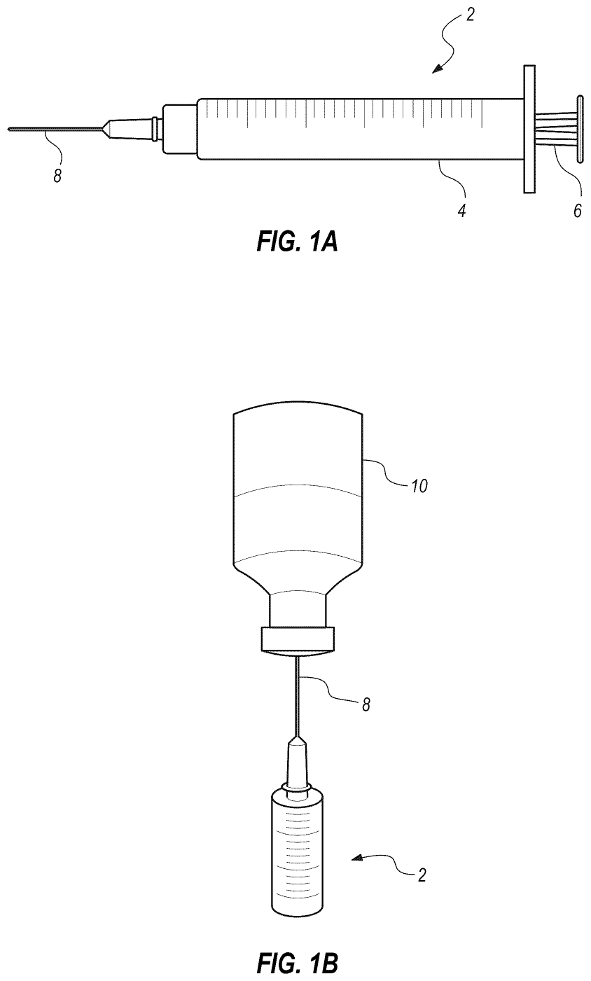

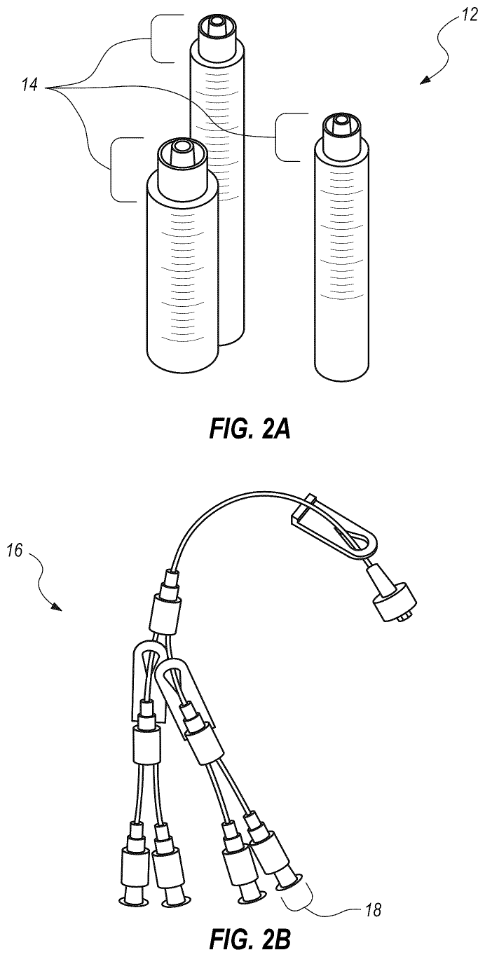

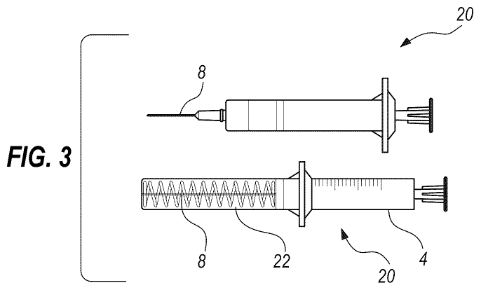

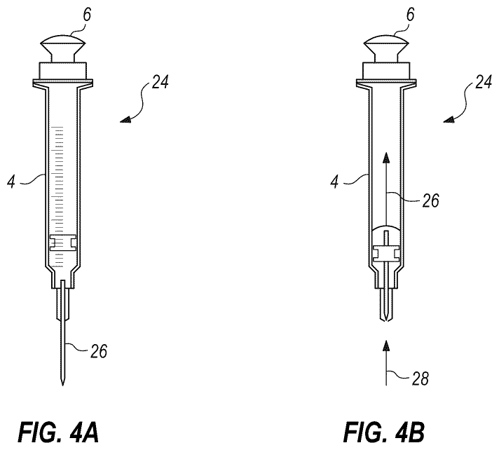

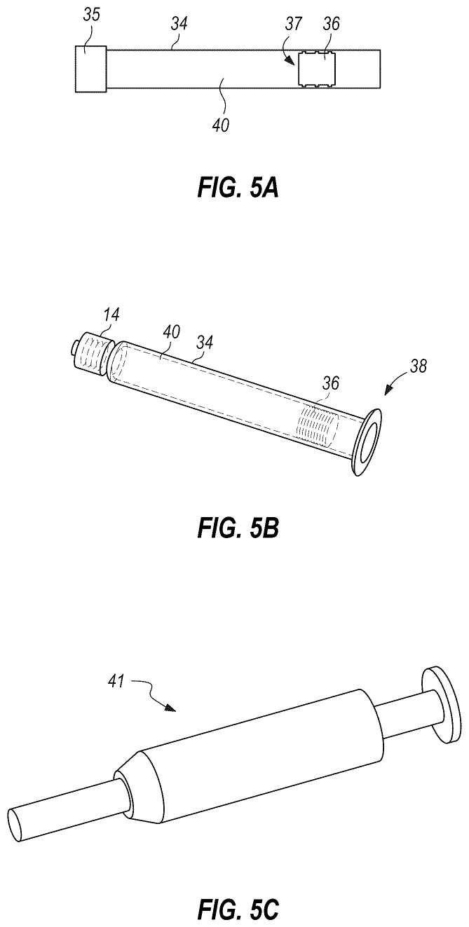

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

### Exemplary Serial Dosing Systems

Many medications can be administered as an injection or topical application to multiple sites and/or at multiple times on the same patient. Some medical procedures involve serial dosing of fixed volumes (e.g., 0.05 ml and/or microliter range volumes) of medications at multiple sites on, or applications to, a patient. Currently, many medicines are drawn into a medicine delivery system from a vial, which increases procedure time and exposure of a sharp distal end of a needle for unintended punctures. Further, some medications are delivered from a medicine tube where accurate microliter dosing is difficult to determine for the patient, and therefore may result in the patient over or under dosing medicine by eye. The serial dosing systems described herein address these shortcomings of current systems.

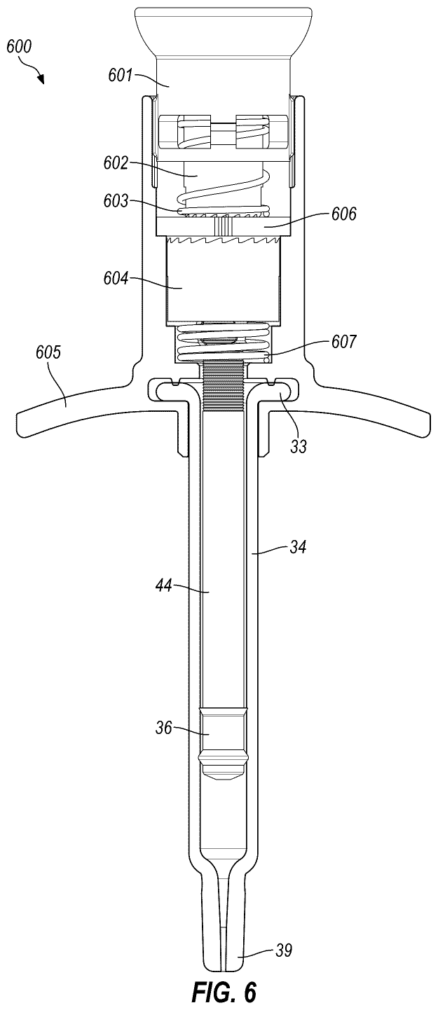

FIGS. 6 to 15B depict a serial dosing system 600 according to some embodiments.

As shown in FIG. 6, the serial dosing system 600 includes a system body 34, a finger flange body 605 coupled to the system body 34, a stopper member 36 disposed in the system body 34, and a plunger member 44 coupled to the stopper member 36. The system body 34 has a system body flange 33 disposed at a proximal end thereof. The finger flange body 605 is coupled to the system body flange 33. The system body 34 also has an open distal end 37. The system body 34 may comprise a syringe or cartridge body. The system body 34 may be constructed of glass or plastic material, which allows for medicine to be pre-filled inside for storage, shipment, and use.

The system 600 also includes a thumbpad member 601 configured to apply distally directed force to the plunger member 44 and the stopper member 36 coupled thereto. The thumbpad member 601 includes a thumbpad tubular member 602 at a distal end thereof. The system 600 further includes a cam 604 disposed slidably in the finger flange body 605 and configured to be both advanced axially and rotated with respect to an inner surface of the finger flange body 605. Moreover, the system 600 includes a ring stator 606 fixedly disposed in the finger flange body 605 proximal of the cam 604.

The finger flange body 605 includes two springs, a proximal spring 603 disposed around the thumbpad tubular member 602 proximal of the ring stator 606 and a distal spring 607 disposed at a distal end of the interior of the finger flange body 605 distal of the cam 604. The proximal spring 603 is configured to apply a distally directed force on the ring stator 606 and a proximally directed force on the thumbpad member 601. The distal spring 607 is configured to apply a proximally directed force on a distal surface of the cam 604. Under the action of the proximal and distal springs 603, 607, the thumbpad member 601, ring stator 606, and cam 604 are generally pushed proximally within the finger flange body 605 by the distal spring 607 and the proximal spring 603.

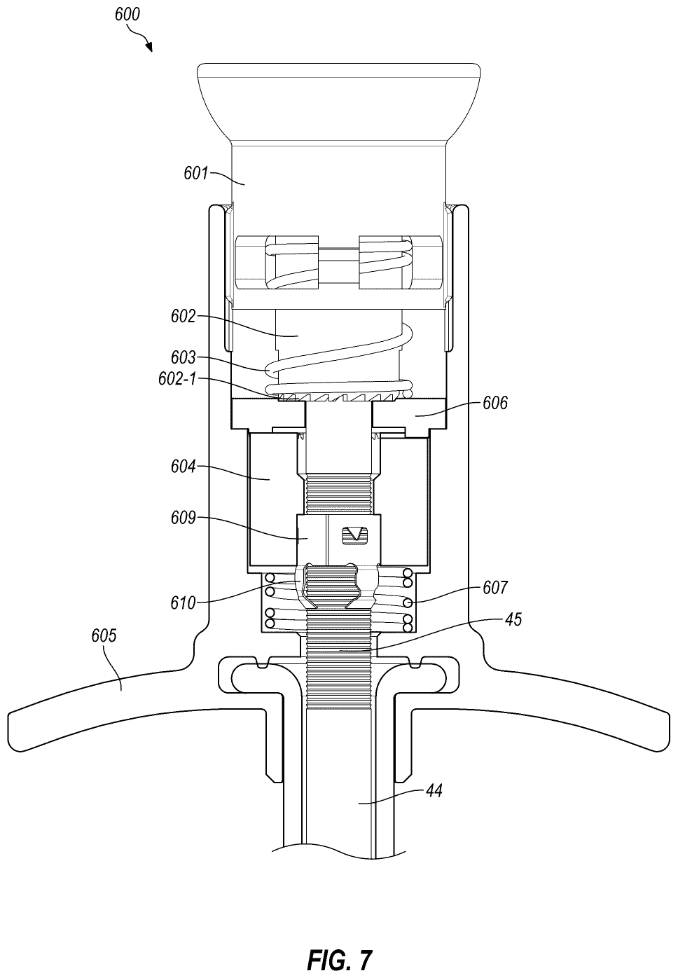

FIG. 7 is a detailed longitudinal cross-sectional view of the serial dosing system 600 depicted in FIG. 6. As shown in FIG. 7, a ratchet 609 is disposed partially within and coupled to the cam 604. The ratchet 609 in the cam member 604 includes a plurality of arms 610 that taper in a distal direction. As such, when the cam 604 moves distally, the plurality of arms 610 on the ratchet 609 push a rib 45 and the plunger member 44 attached thereto in a distal direction. When the cam 604 is returned proximally by the distal spring 607, the plurality of arms 610 on the ratchet 609 can ride over the rib 45 of the plunger member 44 and move in a proximal direction without moving the plunger member. Advancing the plunger member 44 ejects a predetermined/metered dose of fluid or gel disposed in and configured to be delivered from the system body 34. The size of the predetermined/metered dose of fluid or gel can be tuned by modifying the spacing of the ribs 45 on the plunger member 44. Returning the ratchet 609 and the plurality of arms 610 thereof to a position proximal of and adjacent to the next rib 45 of the plunger member 44, and prepares the system 600 to eject the next dose of fluid or gel in a serial manner.

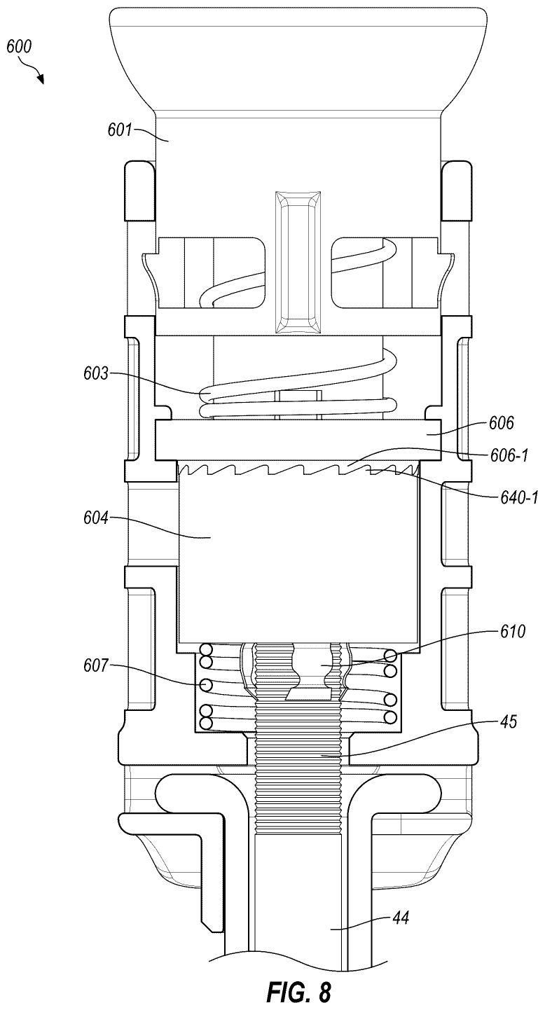

FIG. 8 is a detailed longitudinal cross-sectional view, taken along a plane orthogonal to the cross-section plane in FIG. 7, of the serial dosing system 600 depicted in FIGS. 6 and 7. As shown in FIG. 8, a proximal end of the cam 604 and the distal surface of the ring stator 606 each have respective matching pluralities of interlocking teeth 604-1, 606-1. The distal spring 607 applies a proximally directed force on the cam 604 and maintains pressure on the interface between the cam 604 and the ring stator 606. This pressure induces the respective matching pluralities of interlocking teeth 604-1, 606-1 to remain interlocked with one another as shown in FIG. 8.

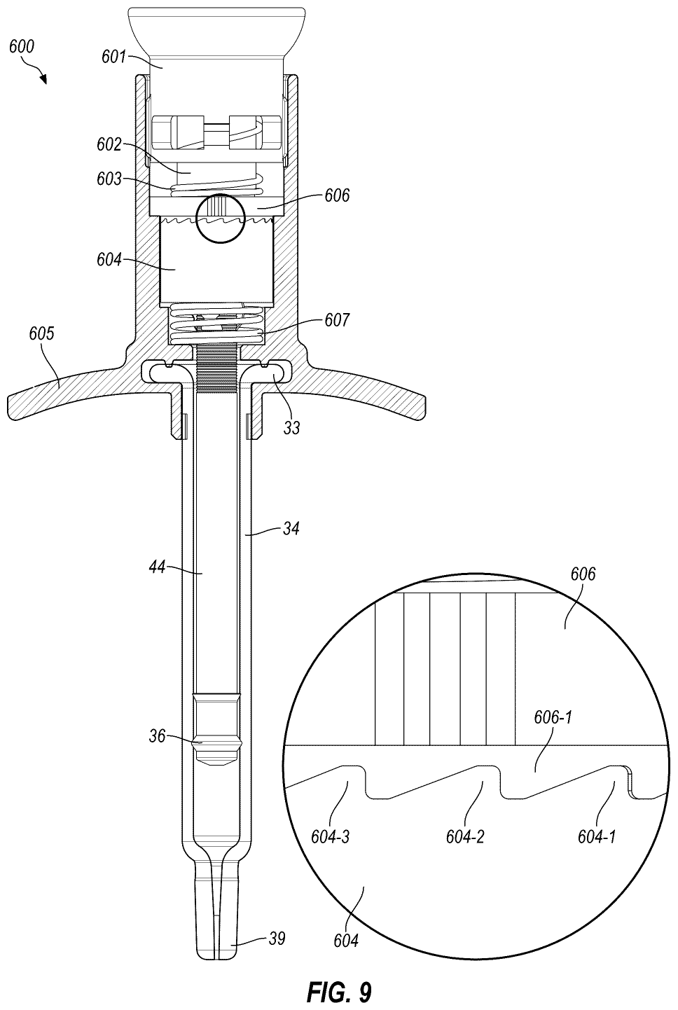

FIGS. 9 to 12 depict an exemplary method of operating a serial dosing system 600 to advance a plunger member 44 to eject a predetermined dose of fluid or gel from a system body 34 while preparing the system 600 to eject the next dose of fluid or gel in a serial manner. FIGS. 9 to 12 are longitudinal cross-sectional views, taken along the same cross-section plane as FIG. 7, of the serial dosing system 600 depicted in FIGS. 6 to 8. In FIGS. 9 to 12, system components have identical reference numbers as those shown in FIGS. 6 to 8. FIGS. 9 to 12 also include respective detailed insets showing interactions between a plurality of interlocking teeth 604-1, 604-2, 604-3 at the proximal end of the cam 604 and a matching plurality of interlocking teeth 606-1 at the distal end of the ring stator 606 at the distal end of the ring stator 606 at various steps during the method.

FIG. 9 is a detailed longitudinal cross-sectional view of the serial dosing system 600 depicted in FIGS. 6 to 8 in a retracted/proximal/ready state. The detailed inset in FIG. 9 shows the matching pluralities of interlocking teeth 604-1, 604-2, 604-3, 606-1. With the device is in its retracted/proximal/ready state, no pressure applied to the thumbpad member 601. As such the cam 604 is rotationally coupled with the ring stator 606 by the matching pluralities of interlocking teeth 604-1, 604-2, 604-3, 606-1. For instance, tooth 604-2 on the cam 604 interferes with tooth 606-1 on the ring stator 606 prevent rotation of the cam 604 and the ring stator 606 in one direction.

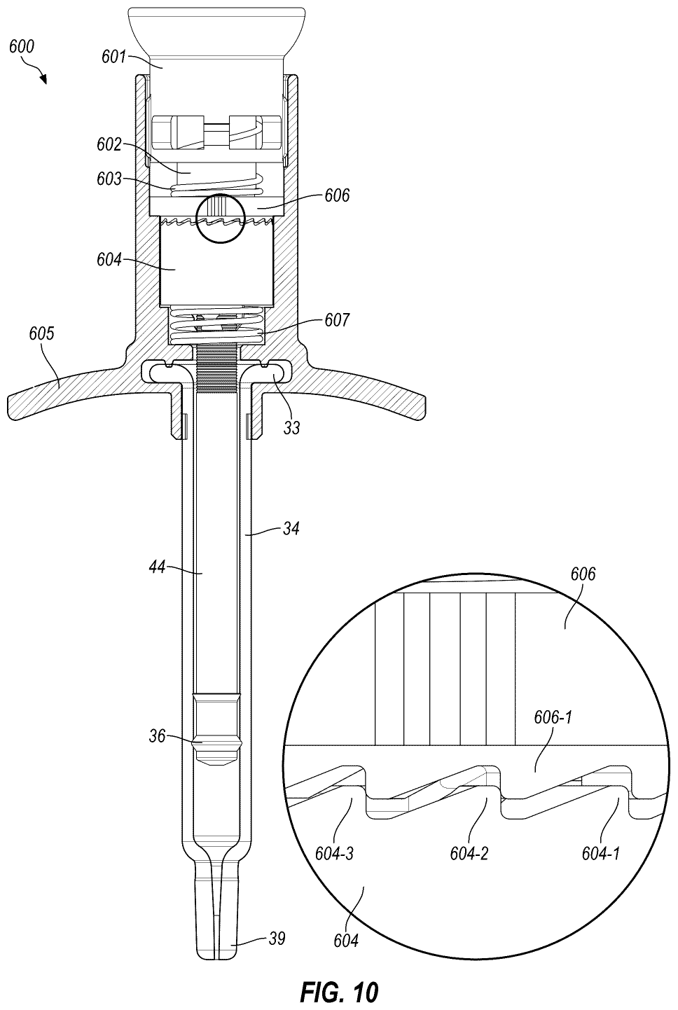

FIG. 10 is a detailed longitudinal cross-sectional view of the serial dosing system 600 in a start dose delivery state. A user can change the serial dosing system 600 from the retracted/proximal/ready state depicted in FIG. 9 to the start dose delivery state depicted in FIG. 10 by applying a distally directed force to the thumbpad member 601. In FIG. 10, the thumbpad member 601 is advanced distally, but not to its most distal position. As such, the distal end of the thumbpad tubular member 602 of the thumbpad member 601 has just come into contact with a proximal surface of the cam 604 and begun moving the cam 604 distally away from the ring stator 606. As shown in the inset in FIG. 10, the cam 604 has started to move distally away from the ring stator 606. Any further distal movement of the thumbpad tubular member 602 of the thumbpad member 601 will necessarily cause the cam 604 to move distally along the thumbpad tubular member 602 in contact therewith. In the start dose delivery state, the thumbpad member 601 not fully advanced to its most distal position, the tubular member of the thumbpad 602 had not advanced to its most distally allowable position. As such the cam 604 has not moved to its most distal position, and the cam 604 remains in contact with the ring stator 606 preventing relative rotation of the cam 604 and the ring stator 606.

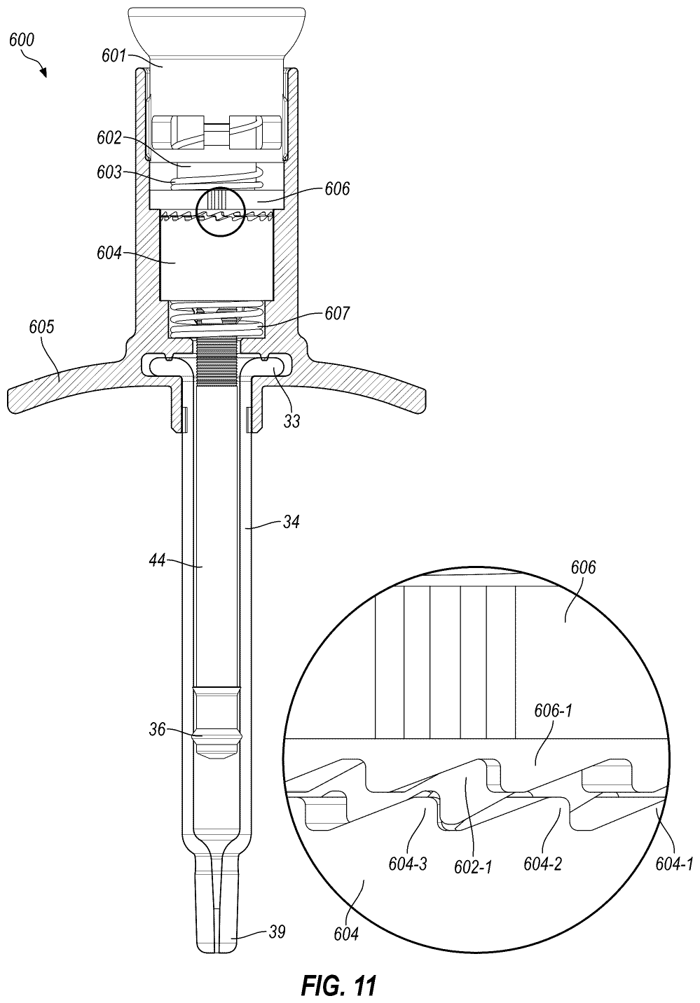

FIG. 11 is a detailed longitudinal cross-sectional view of the serial dosing system 600 in a complete dose delivery state. A user can change the serial dosing system 600 from the start dose delivery state depicted in FIG. 10 to the complete dose delivery state depicted in FIG. 11 by continuing to apply a distally directed force to the thumbpad member 601. In the complete dose delivery state, continued application of the distally directed force to the thumbpad member 601 advances the thumbpad member 601 and the thumbpad tubular member 602 to their respective most distal positions. Because the thumbpad tubular member 602 is in contact with the cam 604, advancing the thumbpad tubular member 602 its most distal position also advances the cam 604 its distal most position. As shown in the inset in FIG. 11, distal advancement of the cam 604 has caused the teeth 604-1, 604-2, 604-3 of the cam 604 to advance distally passed the teeth 606-1 the ring stator 606, and to no longer be in contact/interference therewith. As shown in the inset in FIG. 11, in the complete dose delivery state, the teeth 604-1, 604-2, 604-3 of the cam 604 are positioned and configured to interact with the teeth 602-1 at the distal end of the thumbpad tubular member 602 of the thumbpad member 601 (see FIG. 7), which is circumferentially inside of the cam 604 and the ring stator 606. The teeth 604-1, 604-2, 604-3, 602-1 of the cam 604 and thumbpad tubular member 602 are rotationally offset such that the proximally directed force from the distal spring 607 causes the cam 604 to move proximally and rotate clockwise when viewed from a distal end of the serial dosing system 600. Rotation of the cam 604 is caused/controlled by the relative pitches/tapering of these teeth 604-1, 604-2, 604-3, 602-1. The proximal and rotational movement of the cam 604 may generate an audible indicator as the cam 604 interacts with teeth 602-1 of the thumbpad tubular member 602. The cam 604 and the thumbpad tubular member 602 are configured to generate the audible indicator when the dose delivery system 600 is in the complete dose delivery state, thereby providing an audible indicator of the completion of a dose delivery to the user.

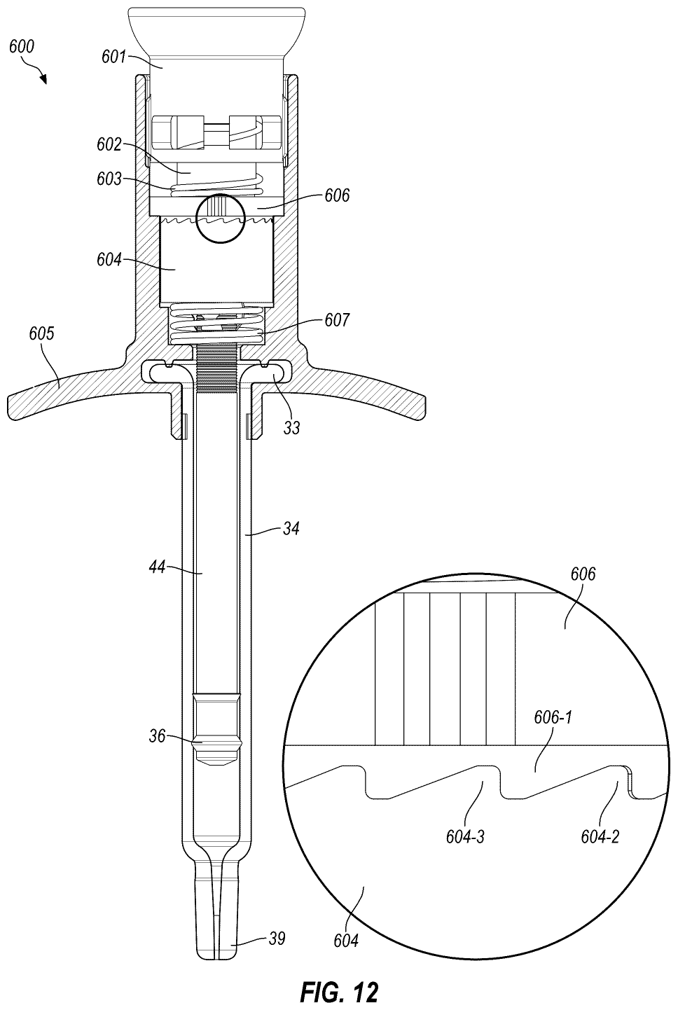

FIG. 12 is a detailed longitudinal cross-sectional view of the serial dosing system 600 in a thumbpad retracted/proximal/ready state. A user can change the serial dosing system 600 from the complete dose delivery state depicted in FIG. 11 to the thumbpad retracted/proximal/ready state depicted in FIG. 12 by releasing the distally directed force from the thumbpad member 601. Releasing the distally directed force from the thumbpad member 601 allows the distal spring 607 to push the cam 604 proximally and back into contact with the ring stator 606. The teeth 606-1 of the ring stator 606 and the teeth 604-1, 604-2, 604-3 of the cam 604 interact with each other such that this proximal movement of the cam 604 will also cause clockwise rotation of the cam 604 along with the interaction between the teeth 604-1, 604-2, 604-3 of the cam 604 and the teeth 602-1 of the thumbpad tubular member 602. This amount of rotation caused by the interaction between the teeth 604-1, 604-2, 604-3, 606-1 of the cam 604 and the ring stator 606 is caused/controlled by the relative pitches/tapering of these teeth 604-1, 604-2, 604-3, 606-1. When the serial dosing system 600 is in the thumbpad retracted/proximal/ready state, the proximal spring 603 has resets the thumbpad member 601 back to its retracted/proximal/ready position depicted in FIGS. 9 and 12. The only differences between the states of the serial dosing system 600 depicted in FIGS. 9 and 12 are that: (1) the cam 604 has rotated one tooth 604-1, 604-2, 604-3 clockwise; and (2) one dose of the fluid or gel has been ejected from the open distal end 37 of the system body 34 by the advancing stopper member 36.

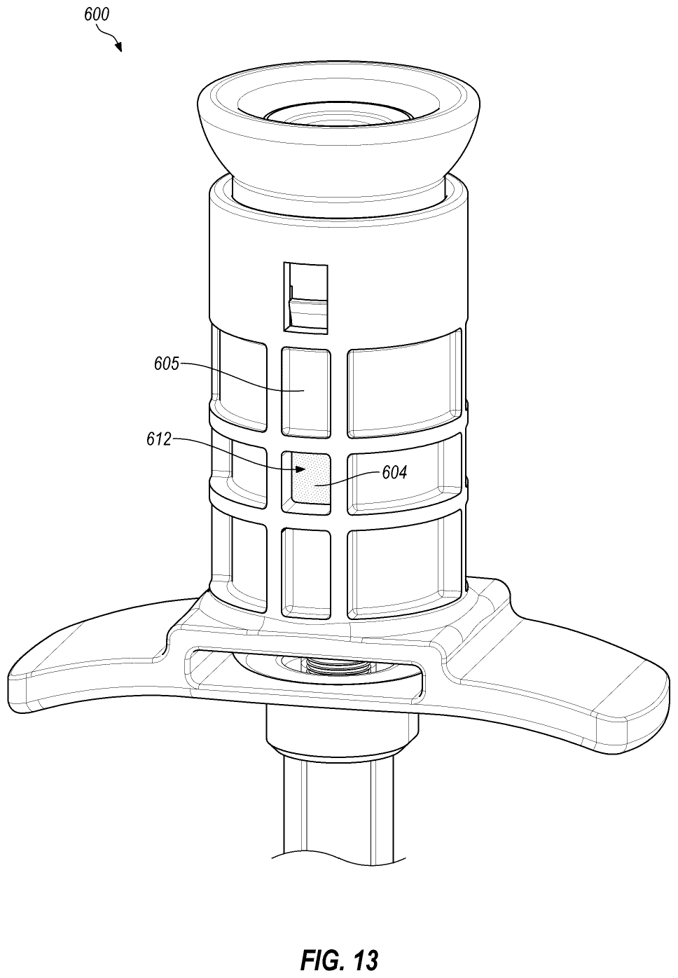

FIG. 13 is a perspective view of a proximal end of a serial dosing system 600 according to some embodiments, such as the serial dosing system 600 depicted in FIGS. 6 to 12. The serial dosing system 600 includes a finger flange body 605 defining an indicator window 612 through which a portion of the cam 604 is visible. As the cam 604 rotates during a sequence of dose deliveries, different portions of the cam 604, which function as visible indicators 614, are visible through the indicator window 612. Accordingly, a plurality of visible indicators 614 can be disposed on the cam 604 to signal a rotational position of the cam 604 and an approximate number of doses remaining or delivered.

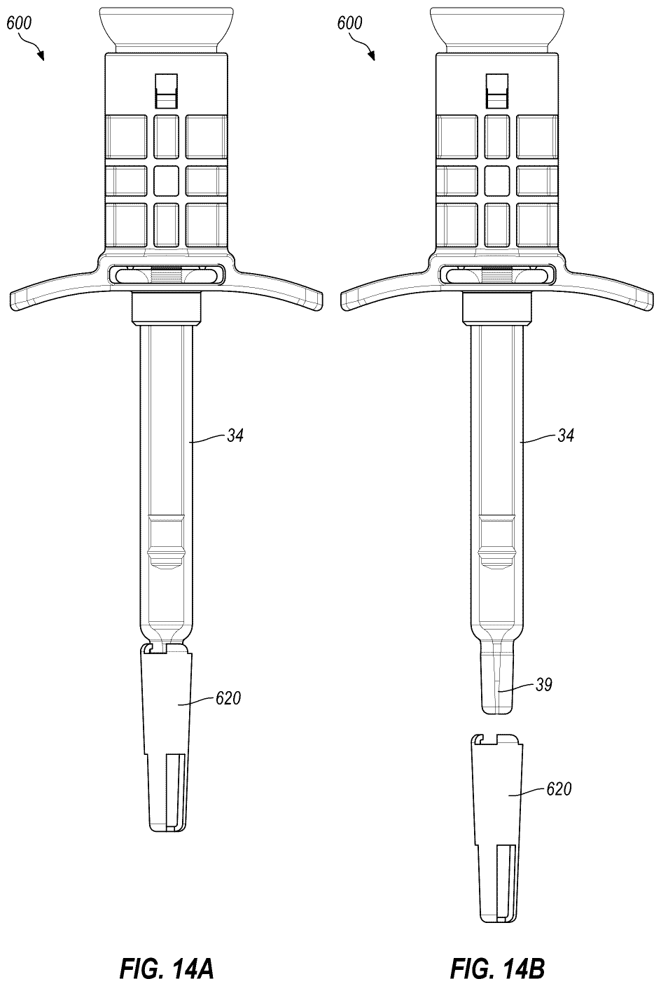

FIGS. 14A and 14B are side views of a multiple site dosing system including a removable cap 620 according to some embodiments, such as the serial dosing system 600 depicted in FIGS. 6 to 13. When the removable cap 620 is coupled to the system body 34 as shown in FIG. 14A, it protects the open distal end 37 (see FIG. 14B) from contamination. The removable cap can also protect against accidental delivery of the fluid or gel.

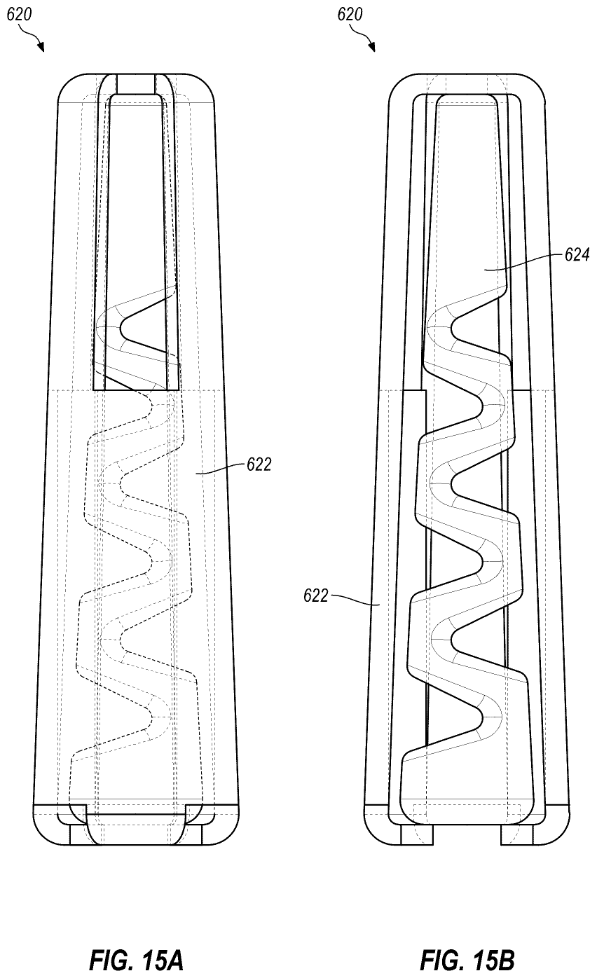

FIGS. 15A and 15B are side and longitudinal cross-sectional views of a removable cap 620 according to some embodiments. The removable cap 620 includes a rigid outer shell 622 and a tortuous elastic liner 624 (see FIG. 15B) disposed in the rigid outer shell 622 to secure the removable cap 620 onto the open distal end 37 of the system body 34 (see FIG. 14B).

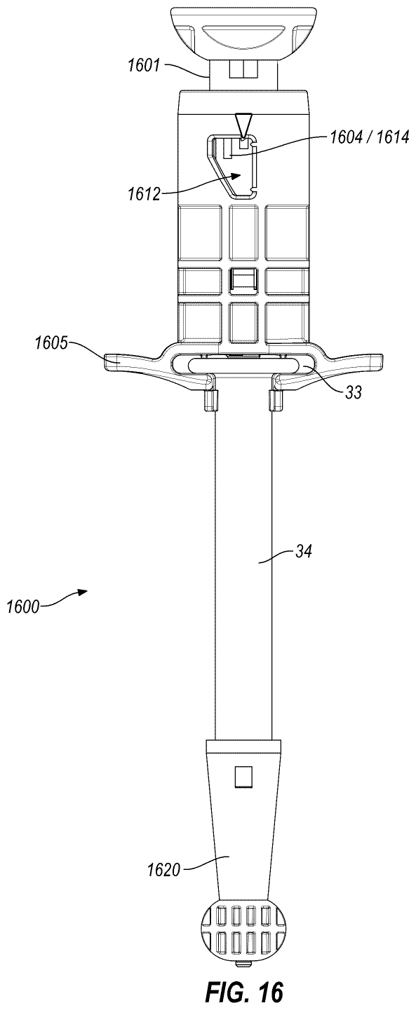

FIGS. 16 to 37 depict a serial dosing system 1600 having a single helical spring 1607 according to some embodiments. The serial dosing system 1600 depicted in FIGS. 16 to 37 is similar to the serial dosing system 600 depicted in FIGS. 6 to 15, and similar components have identical or similar reference numbers.

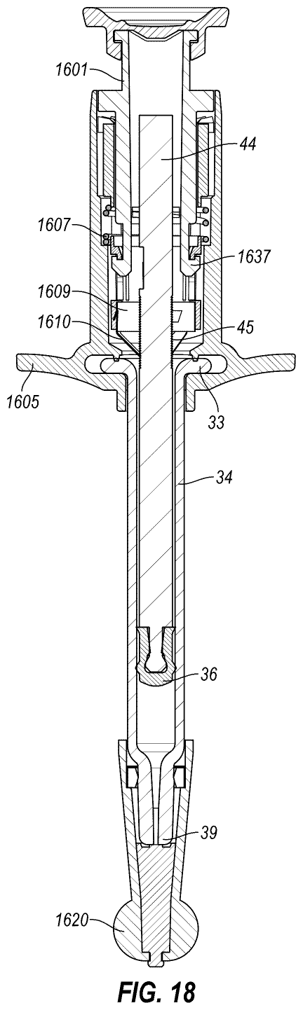

FIG. 16 is a side view depicting the serial dosing system 1600 according to some embodiments. The serial dosing system 1600 includes a system body 34, a finger flange body 1605 coupled to the system body 34, and a removable cap 1620 coupled to a distal end of the system body 34. The system body 34 has a system body flange 33 disposed at a proximal end thereof. The system body 34 also has an open distal end 37 as shown in FIG. 18. The system body 34 may comprise a syringe or cartridge body. The system body 34 may be constructed of glass or plastic material, which allows for medicine to be pre-filled inside for storage, shipment, and use.

The finger flange body 1605 is coupled to the system body flange 33. The finger flange 1605 defines an indicator window 1612 through which a portion of a cam 1604, which functions as a visible indicator 1614, is visible. The system 1600 also includes a thumbpad member 1601 configured to apply distally directed force to the serial dosing system 1600.

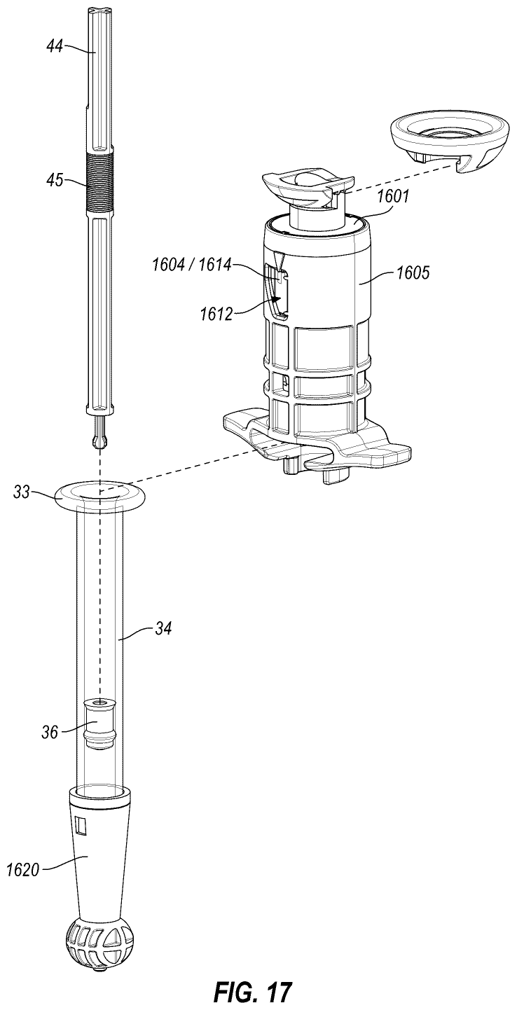

FIG. 17 is a partially exploded view of various components from the serial dosing system 1600 depicted in FIG. 16 according to some embodiments. In addition to the components of the serial dosing system 1600 described above, FIG. 17 depicts a stopper member 36 disposed in the system body 34. A plunger member 44 is also depicted having a plurality of ribs 45 disposed thereon. Moreover, a finger flange 1605 is depicted along with a thumbpad member 1601 disposed therein.

FIG. 18 is a longitudinal cross-sectional view of the serial dosing system 1600 depicted in FIG. 16. In addition to the components of the serial dosing system 1600 described above, FIG. 18 depicts a helical spring 1607 disposed around a distal end of the thumbpad member 1601 and a portion of the plunger member 44. FIG. 18 also depicts a ratchet 1609 disposed in the finger flange 1605 and configured to be moved distally by the thumbpad member 1601. The ratchet 1609 has a plurality of arms 1610 that taper in a distal direction. As such, when the thumbpad member 1601 moves distally, the plurality of arms 1610 on the ratchet 1609 push a rib 45 and the plunger member 44 attached thereto in a distal direction to ejects a predetermined/metered dose of fluid or gel from the system body 34.

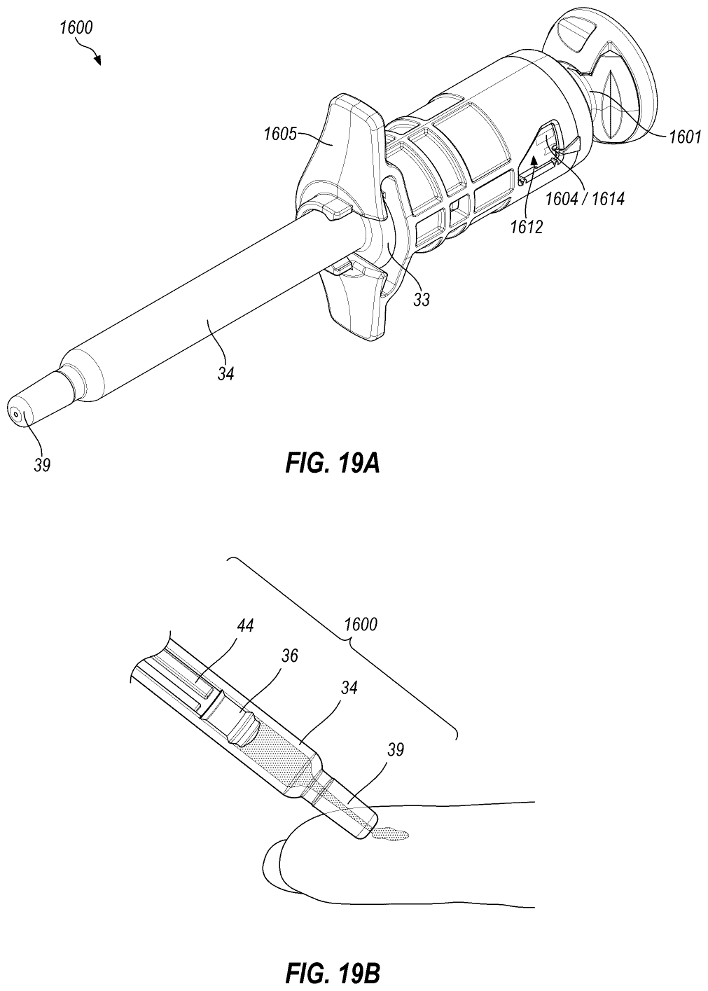

FIG. 19A is a front perspective view of the serial dosing system 1600 depicted in FIG. 16 with the cap 1620 (see FIG. 16) removed according to some embodiments. FIG. 19B schematically depicts the distal end of the serial dosing system 1600 depicted in FIG. 16 with the cap 1620 (see FIG. 16) removed dispensing a fluid or gel onto a finger of a user (e.g., for topical application) according to some embodiments.

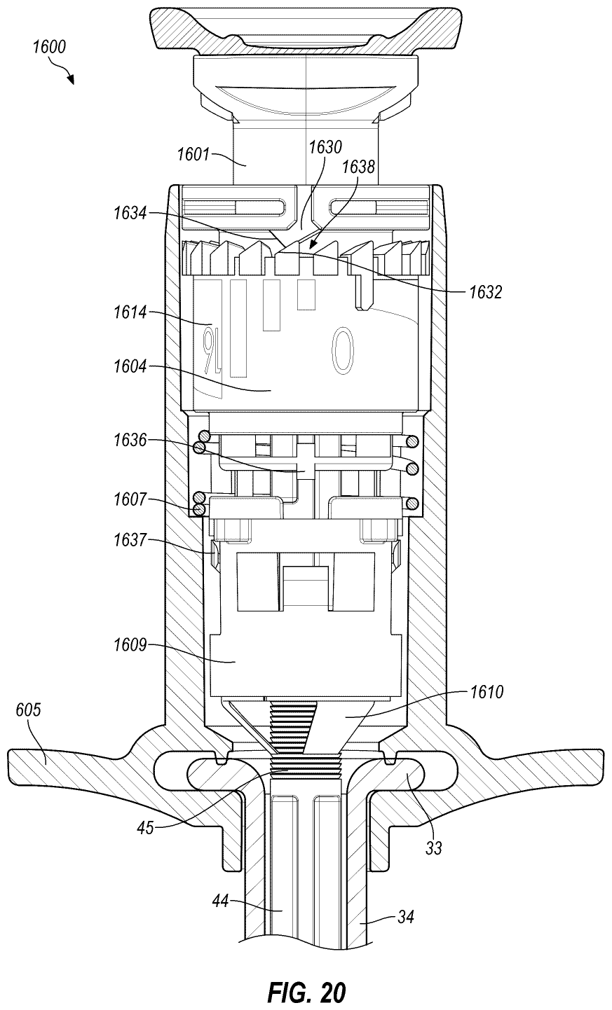

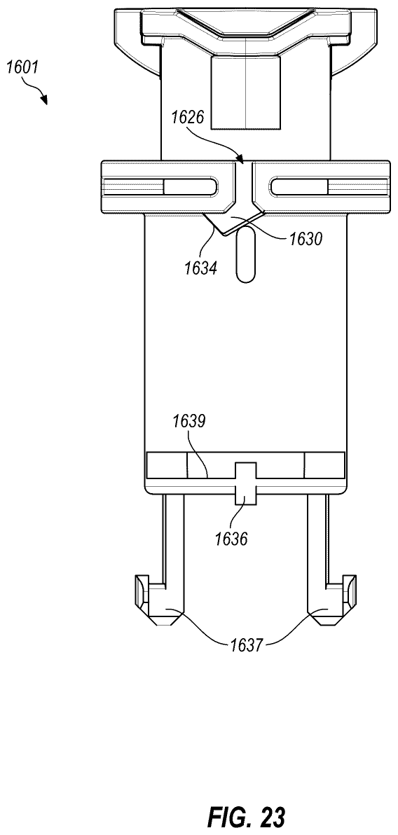

FIGS. 20 to 22 are a detailed longitudinal cross-sectional view of the serial dosing system depicted in FIG. 16 during the delivery of a single dose of fluid or gel. In addition to the components of the serial dosing system 1600 described above, FIG. 20 also depicts the thumbpad member 1601 in more detail, including a thumbpad rotation ramp 1630, a stop surface 1634, a ratchet contact patch 1636, and a pair of hooks 1637 (see also FIG. 23). As shown in FIG. 23, the thumbpad member 1601 also defines a pair of stator notches 1626 (only one shown).

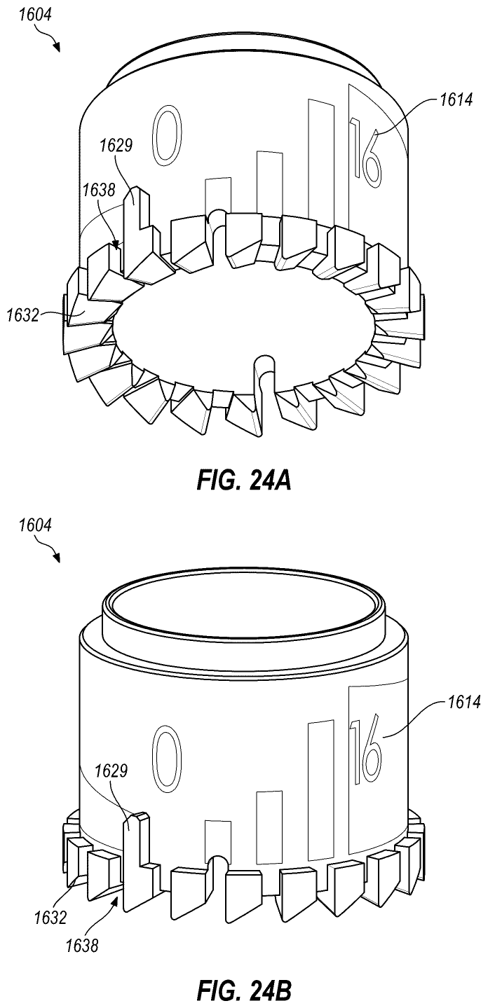

FIG. 20 also depicts the cam 1604 in more detail, including respective pluralities of cam rotation ramps 1632 and visible indicators 1614 (see also FIGS. 24A and 24B). FIGS. 24A and 24B further depict a cam lockout rib 1629 and a plurality of stator bypass grooves 1638.

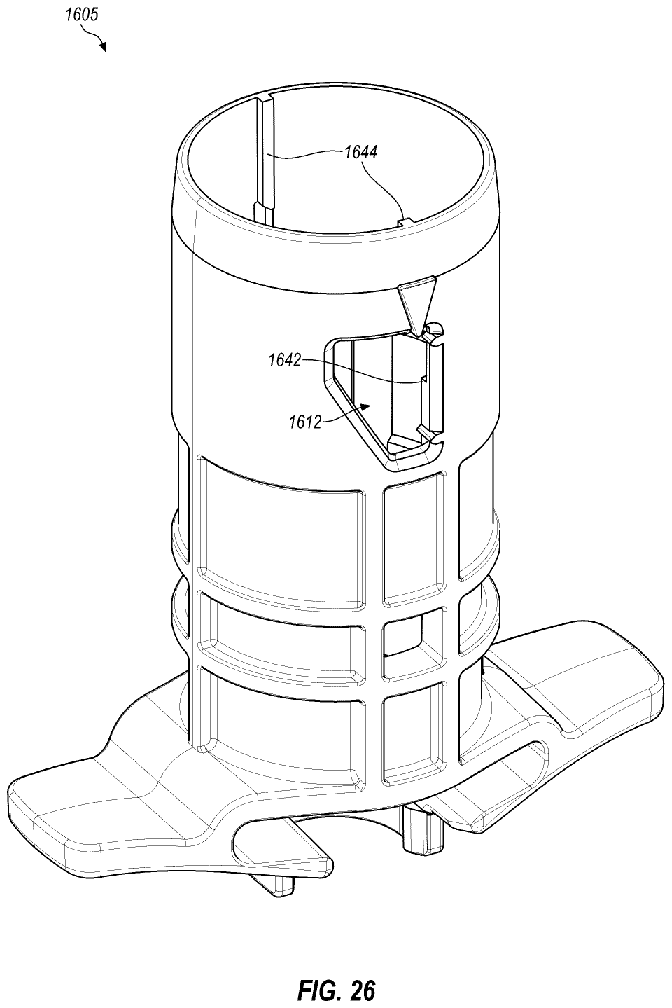

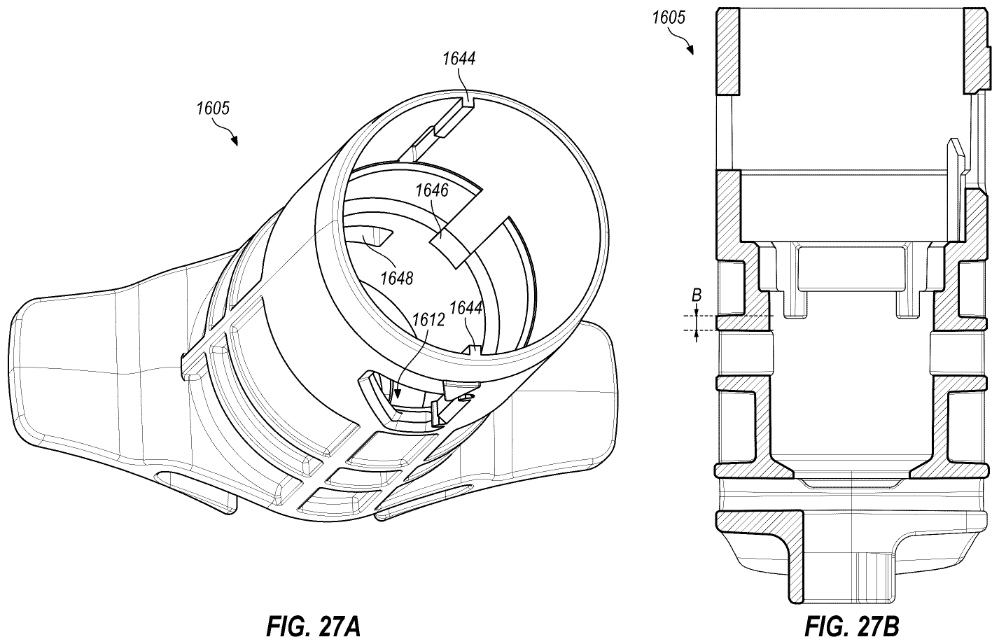

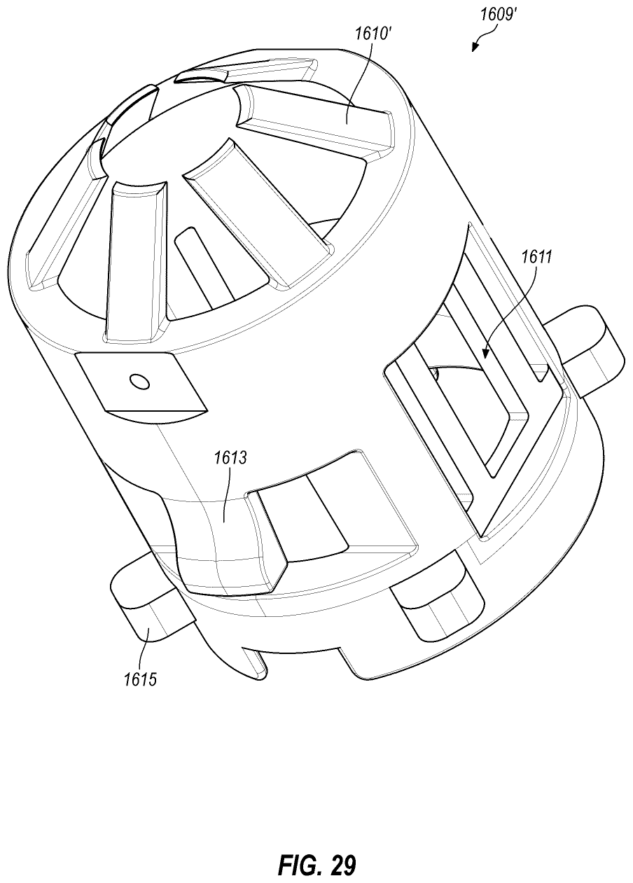

FIG. 20 depicts the serial dosing system 1600 in a retracted/proximal/ready state in which it is ready to deliver a dose of fluid or gel. In the retracted/proximal/ready state, the helical spring 1607 is expanded, thereby pushing the cam 1604 proximally such that a cam rotation ramp 1632 is disposed against the thumbpad rotation ramp 1630 and the cam bypass grooves 1638 (see FIGS. 24A and 24B) are in contact with the stator ribs 1644 (see FIG. 26). Further, the ratchet contact patch 1636 is not in contact with the ratchet 1609. The helical spring 1607 pushes the cam 1604 and thumbpad member 1601 proximally, which causes the intercoupled ratchet 1609 to be pulled proximally via hooks 1637 until retention tabs 1613 on the ratchet 1609 (see FIG. 29B) are in contact with a proximal surface of the retention slot 1648 defined by the finger flange 1605 (see FIG. 27A).

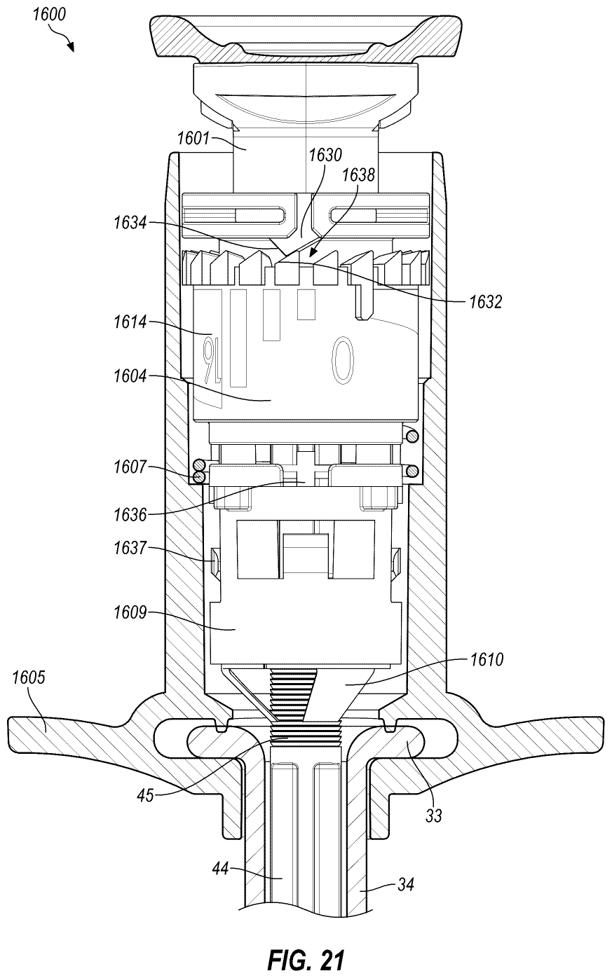

FIG. 21 depicts the serial dosing system 1600 in a start dose delivery state. A user can change the serial dosing system 1600 from the retracted/proximal/ready state depicted in FIG. 20 to the start dose delivery state depicted in FIG. 21 by applying a distally directed force to the thumbpad member 1601. In FIG. 21, the thumbpad member 1601 is advanced distally, but not to its most distal position. As such, the ratchet contact patch 1636 of the thumbpad member 1601 has just come into contact with a proximal surface of the ratchet 1609 and begun moving the ratchet 1609 distally. In the start dose delivery state, the thumbpad member 1601 not fully advanced to its most distal position. As such the cam 1604 and the ratchet 1609 have not moved to their most distal positions. Any further distal movement of the thumbpad member 1601 will necessarily cause the cam 1604 and the ratchet 1609 to move distally.

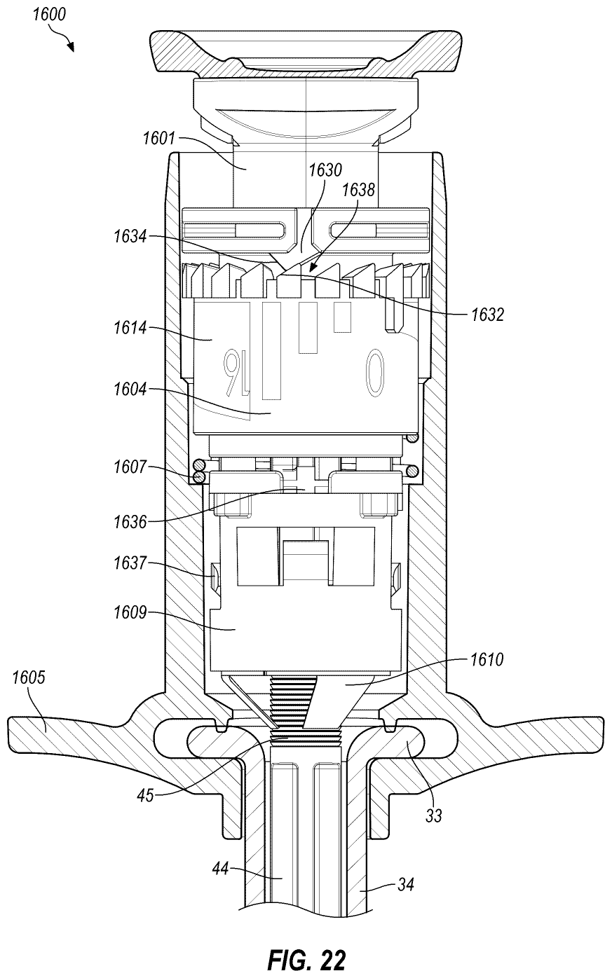

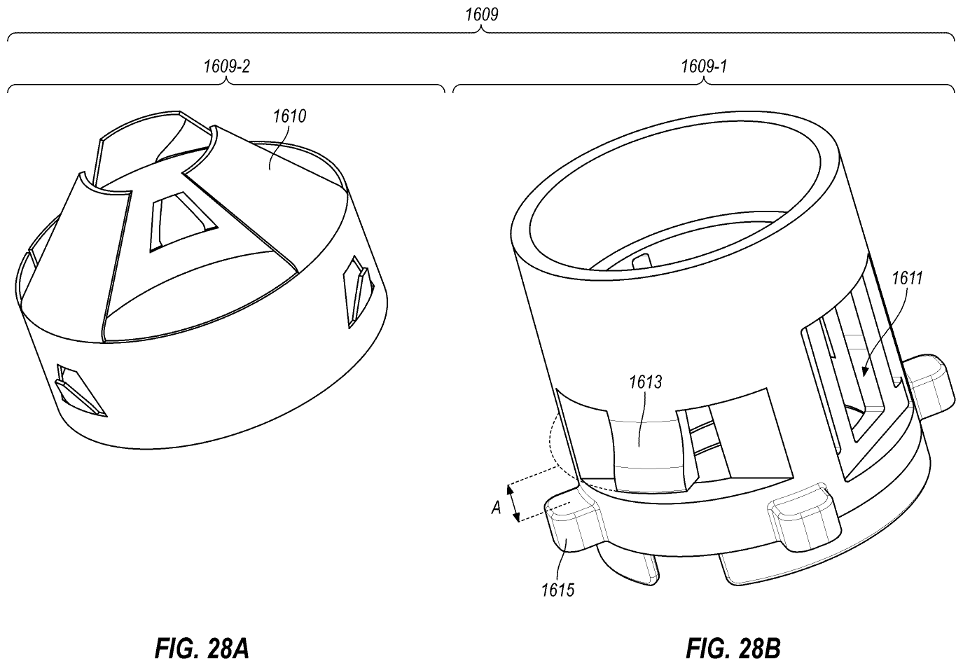

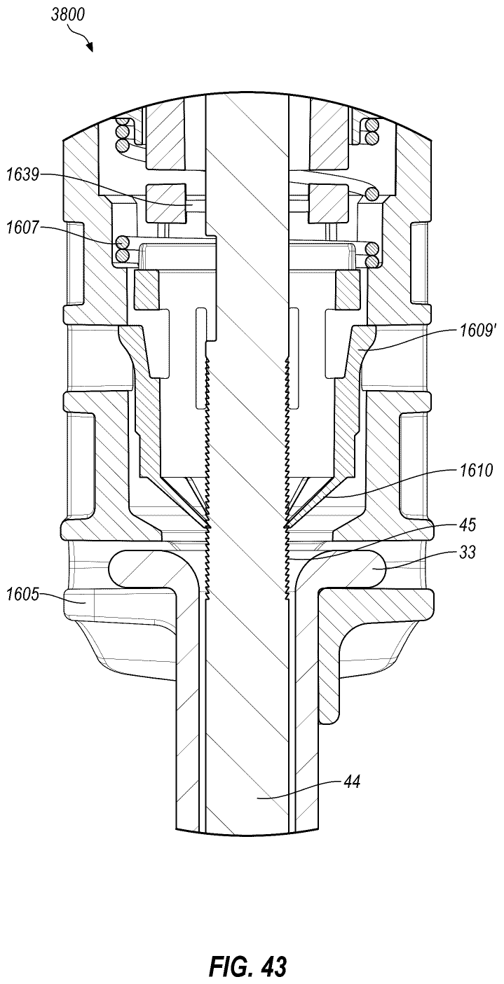

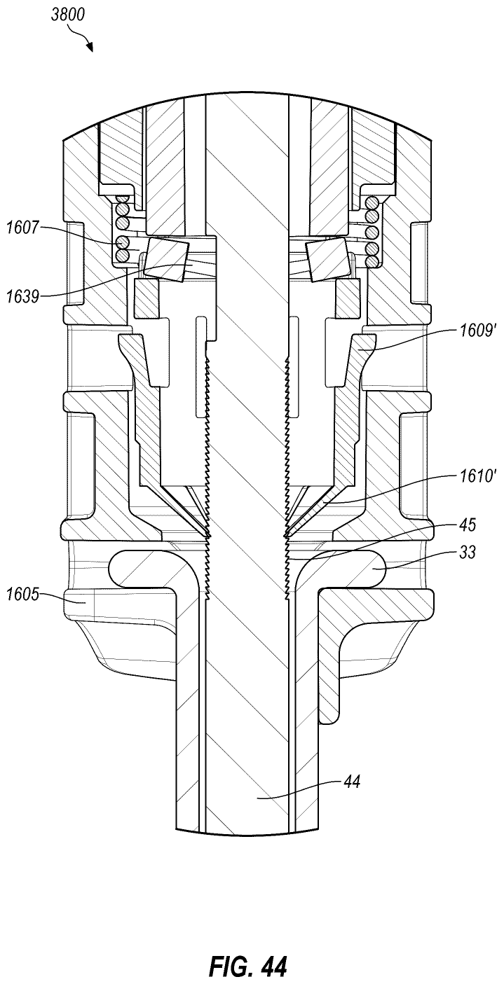

FIG. 22 depicts the serial dosing system 1600 in a complete dose delivery state. A user can change the serial dosing system 1600 from the start dose delivery state depicted in FIG. 21 to the complete dose delivery state depicted in FIG. 22 by continuing to apply a distally directed force to the thumbpad member 1601. In the complete dose delivery state, continued application of the distally directed force to the thumbpad member 1601 advances the thumbpad member 1601, the cam 1604, and the ratchet 1609 to their respective most distal positions. Distal movement of the ratchet 1609 is stopped by ratchet stop tabs 1615 (see FIG. 28B) reaching stop channel bottom surface 1646 defined by the finger flange 1605 (see FIG. 27A). Continued application of the distally directed force to the thumbpad member 1601 compresses leaf springs 1636 (see FIGS. 23, 43, and 44) in the thumbpad member 1601 allowing the cam 1604 to further translate distally until the cam bypass grooves 1638 are clear of the stator ribs 1644, thereby allowing rotation of the cam 1604 in a clockwise direction when viewed from a distal end of the serial dosing system 1600. Further translation of the cam 1604 facilitated by compression of the leaf springs 1636, provides tolerance to various system components that couple the cam 1604 and the ratchet 1609. The further translation of the cam 1604 also requires extra force from user (e.g., via the user's thumb) to generate a sense of false motion during a dose delivery cycle.

Rotation of the cam 1604 is caused by interaction between the thumbpad rotation ramp 1630 and the cam rotation ramp 1632 currently in contact therewith. Rotating the cam 1604 changes the visible indicator 1614 that is visible through the indicator window (not shown, see FIG. 16). Rotating the cam 1604 also generates an audible indicator as the cam rotation ramp 1632 slides with respect to, then collides with, the stop surface 1634 on the thumbpad rotation ramp 1630. The cam 1604 and the thumbpad rotation ramp 1630 are configured to generate the audible indicator when the dose delivery system 1600 is in the complete dose delivery state, thereby providing an audible indicator of the completion of a dose delivery to the user.

Advancing the thumbpad member 1601 to its most distal position also advances the ratchet 1609 and the plurality of arms 1610 thereon to their respective distal most positions using distally directed force delivered via the ratchet contact point 1636 on the thumbpad member 1601. The distally advancing plurality of arms 1610 push a rib 45 and the plunger member 44 attached thereto in a distal direction to ejects a predetermined/metered dose of fluid or gel from the system body 34 to complete a delivery of a predetermined/metered dose of fluid or gel therefrom. The plunger member 44 and intercoupled stopper 36 move distally to define the dose delivered.

The amount of plunger member 44 distal movement is determined by the pitch of the ribs 45 on the plunger member 44. The arms 1610 of the ratchet 1609 are configured to bear against the ribs 45 and move between 1× and 2× the pitch between the ribs 45 (i.e., more than one inter-rib space but less than two inter-rib spaces). The difference between the plunger member 44 movement and the ratchet 1609 movement ensures that after dosing, the ratchet engages the next proximal rib 45 after proximal movement of the plunger member 44 and intercoupled ratchet 1609 and release of the thumbpad member 1601 by the user. The amount of ratchet movement is the difference in dimension between the ratchet stop tabs 1615 to ratchet retention tabs 1613 (i.e., dimension A depicted in FIG. 28B), and the stop channel bottom surface 1646 to retention slot proximal surface 1648 (i.e., dimension B depicted in FIG. 27B). The difference between dimensions A and B (i.e., A-B) is the amount of movement of the ratchet 1609 during one dose delivery cycle. In some embodiments, the pitch for the ribs 45 on the plunger member 44 is on the order of 1/1,000 of an inch.

Releasing the distally directed force to the thumbpad member 1601 (see also FIGS. 18 and 34 to 35) allows the helical spring 1607 to return the thumbpad member 1601 proximally and pulls the ratchet 1609 via the hooks 1637 in a proximal direction, allowing the arms 1610 on the ratchet 1609 to jump to the next proximal rib 45 on the plunger member 44 to reset the serial dosing system 1600 for the next dosing event.

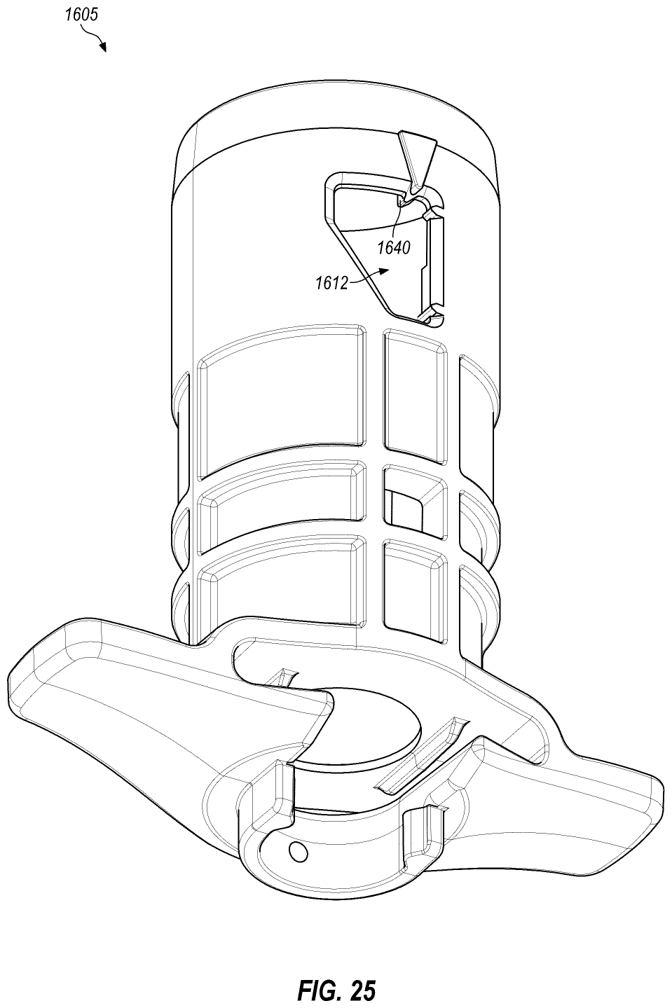

FIGS. 25 to 27 are distal (FIG. 25) and proximal (FIGS. 26 and 27) perspective views of the finger flange 1605. In addition to the components of the serial dosing system 1600 described above, FIG. 25 depicts a stator rotation ramp 1640 extending from a distal end of a stator rib 1644 (see FIGS. 26 and 27) and configured to complete rotation of the cam 1604 at the end of a dose delivery cycle (described below). FIG. 26 depicts a lockout receiver 1642 configured to prevent further movement of the thumbpad member 1601 and rotation of the cam 1604 after a predetermined number of doses have been delivered (described below). FIGS. 26 and 27 also depict a pair of stator ribs 1644 (see FIG. 23) configured to interfere with a corresponding pair of stator notches 1628 to prevent rotation of the thumbpad member 1601 and cam 1604 (in some situations described below) relative to the finger flange 1605.

FIGS. 28A and 28B depict a two-part ratchet 1609 for use with the serial dosing system 1600 according to some embodiments. The two-part ratchet 1609 includes a proximal portion 1609-1, which may be formed from a polymer, and a distal portion 1609-2, which may be formed from a metal/alloy such as stainless steel. The proximal portion 1609-1 of the two-part ratchet 1609 also defines a pair of longitudinal slots 1611 (only one shown) for receiving the corresponding pair of hooks 1637 at the distal end of the thumbpad member 1601. The distal portion 1609-2 of the two-part ratchet 1609 also defines a plurality of arms 1610 as described herein. The arms 1610 are configured to be elastically deformed to fit around an outer diameter of the plunger member 44 and engage the ribs 45 on the plunger member 45 when dispensing a dose of fluid or gel. Upon the delivery of a dose, the ratchet 1609 is configured to be moved proximally by the helical spring 1607 and the arms 1610 snap over the next most proximal rib 45 on the plunger member 44 to engage that next most proximal rib 45 to ready the system 1600 for the next dose to be given. The ratchet 1609 further comprises a plurality of ratchet stop tabs 1615 and ratchet retention tabs 1613.

FIG. 29 depicts an integral ratchet 1609′ for use with the serial dosing system 1600 according to some embodiments. The ratchet 1609′ may be formed from a polymer. The ratchet 1609′ also defines a pair of longitudinal slots 1611 (only one shown) for receiving the corresponding pair of hooks 1637 at the distal end of the thumbpad member 1601. Moreover, the ratchet 1609′ defines a plurality of arms 1610′ as described herein. The arms 1610′ are formed from a polymer and integral to the ratchet 1609′. The arms 1610′ are configured to be elastically deformed to fit around the outside diameter of the plunger member 44 and engage the ribs 45 on the plunger member 45 when dispensing a dose of fluid or gel. The ratchet 1609′ further comprises a plurality of ratchet stop tabs 1615 and ratchet retention tabs 1613.

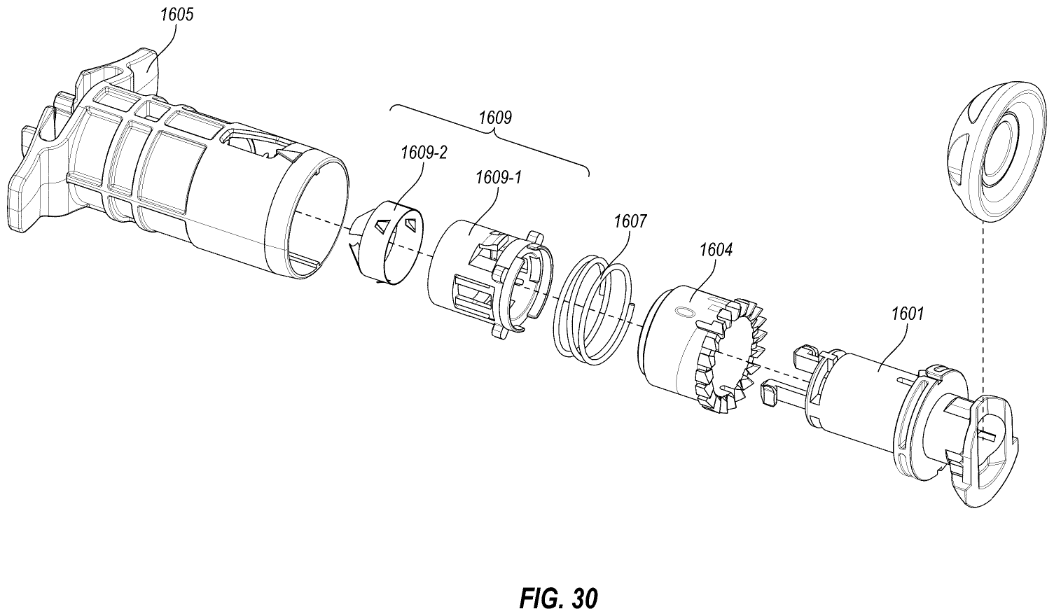

FIG. 30 is an exploded view of the finger flange 1605 of the serial dosing system 1600, and the components contained therein. The components contained in the finger flange 1605 include (from proximal to distal) the thumbpad member 1601, the cam 1604, the helical spring 1607, and the ratchet 1609.

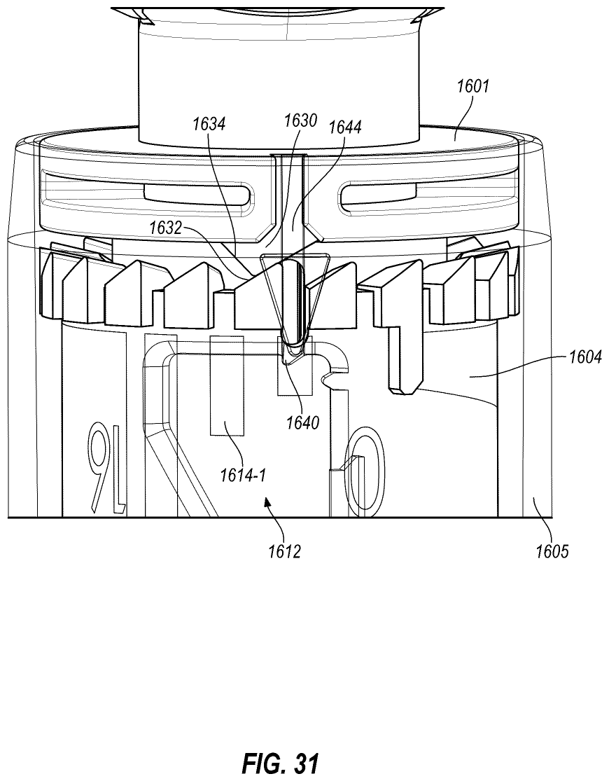

FIGS. 31-35 are side perspective views schematically depicting a sequence of the dose counting mechanism of the serial dosing system 1600 of FIG. 16. The dose counting mechanism includes the cam 1604, which is configured to rotate a predetermined angle with each distal advancement of the thumbpad member 1601 and does delivery. The dose counting mechanism also includes the stator rotation ramp 1640 disposed on an inner surface of the finger flange 1605 (see FIG. 25) and the thumbpad rotation ramp 1630 disposed on an outer surface of the thumbpad member 1601 (see FIG. 23).

FIG. 31 depicts the counting mechanism in a resting state where the cam 1604 is wedged between the thumbpad rotation ramp 1630, the stator rotation ramp 1640, and the stator rib 1644, thereby preventing rotation of the cam 1604.

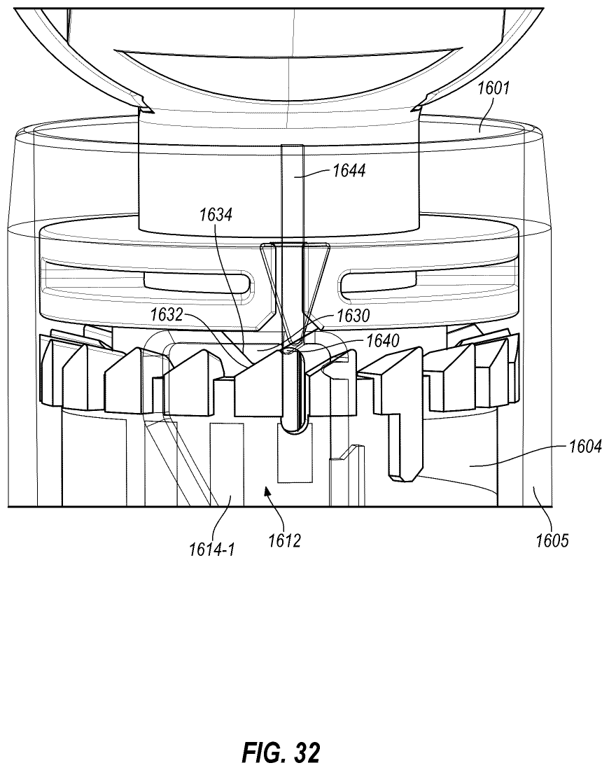

FIG. 32 depicts the counting mechanism after sufficient distally directed force has been applied to the thumbpad member 1601 to advance the thumbpad member 1601 and the cam 1604 out of contact with the stator rotation ramp 1640 and the stator rib 1644. At this point, the counting mechanism has entered a rotation state. In the rotation state, interaction between the thumbpad rotation ramp 1630 and a cam rotation ramp 1632 causes the cam to begin to rotate in a clockwise direction viewed from a distal end of the serial injection device 1600. The dose delivery begins during this phase of the distal movement of the thumbpad member 1601 as the ratchet contact patch 1636 is in contact with the ratchet 1609, through the leaf spring 1639, moving the ratchet 1609 and the intercoupled plunger member 44 and stopper member 36 distally, dispensing the fluid or gel from the system body 34.

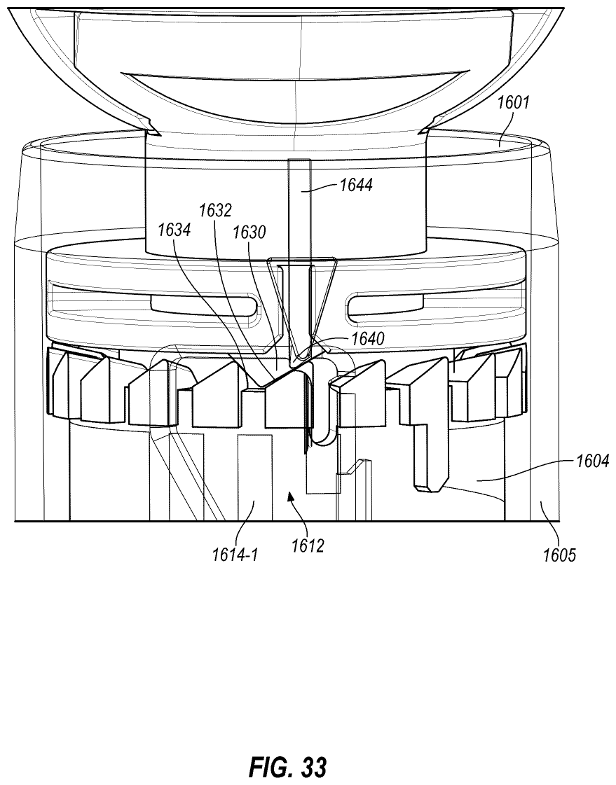

FIG. 33 depicts the counting mechanism after additional distally directed force has been applied to the thumbpad member 1601 to rotate the cam 1604 until the next cam rotation ramp 1632 abuts the stop surface 1634 on the thumbpad rotation ramp 1630. At this point, the cam 1604 has rotated approximately half of the predetermined amount of rotation per dose delivery cycle. The dose dispensing movement is completed at this point.

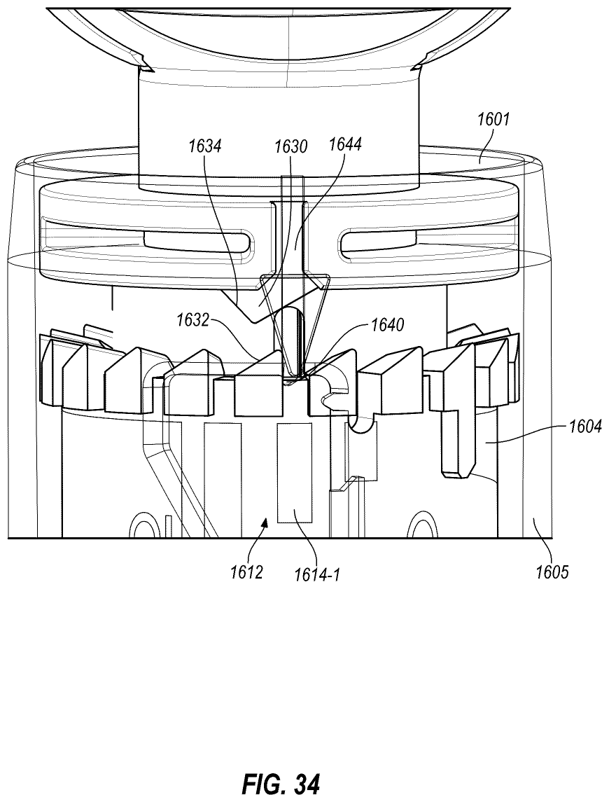

FIG. 34 depicts the counting mechanism after the distally directed force has been removed from the thumbpad member 1601 and the helical spring 1607 (see FIGS. 20 and 30) has begun to return the cam 1604 and the thumbpad member 1601 distally. During the return of the cam 1604 distally the cam rotation ramp 1632 interacts with the stator rotation ramp 1640 to complete rotation of the half rotated cam 1604 for this dose delivery cycle.

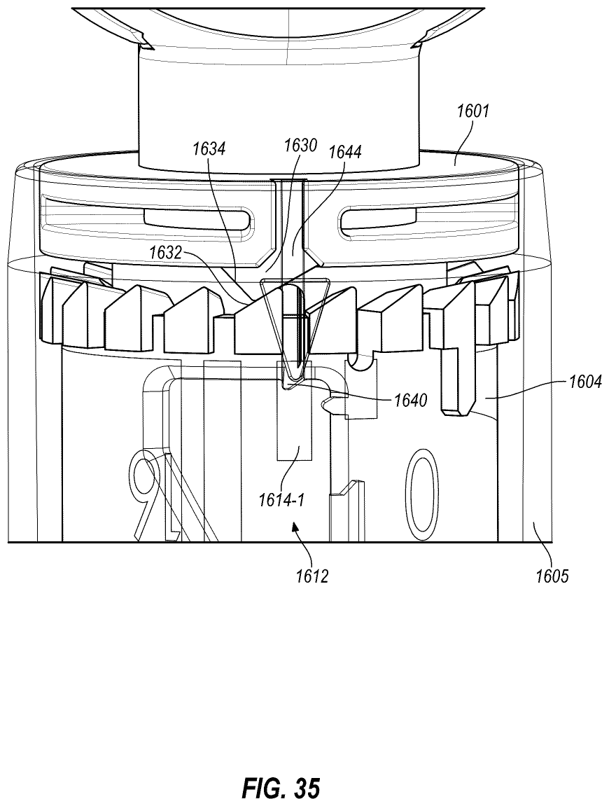

FIG. 35 depicts return of the counting mechanism to the resting state by expansion of the previously compressed helical spring 1607 (see FIGS. 20 and 30). The resting state depicted in FIG. 35 is similar to the resting state depicted in FIG. 31 in that the cam 1604 is wedged between the thumbpad rotation ramp 1630, the stator rotation ramp 1640, and the stator rib 1644, thereby preventing rotation of the cam 1604. However, the cam 1604 has been rotated a predetermined amount clockwise. At this point, the dose delivery cycle is completed and the cam 1604 has been rotated such that the visible indicia 1614-1 is visible through the indicator window 1612 in the finger flange 1605. The amount of cam rotation per dose delivery cycle can be controlled by configuring the relative sizes and shapes of the thumbpad rotation ramp 1630, cam rotation ramps 1632, and stator rotation ramp 1640. Entering this state, the ratchet 1609 is pulled proximally by the helical spring 1607 coupled to the cam 1604 and thumbpad member 1601 via the hooks 1637, causing the ratchet arms 1610 to engage the next most proximal rib 45 on the plunger member 44.

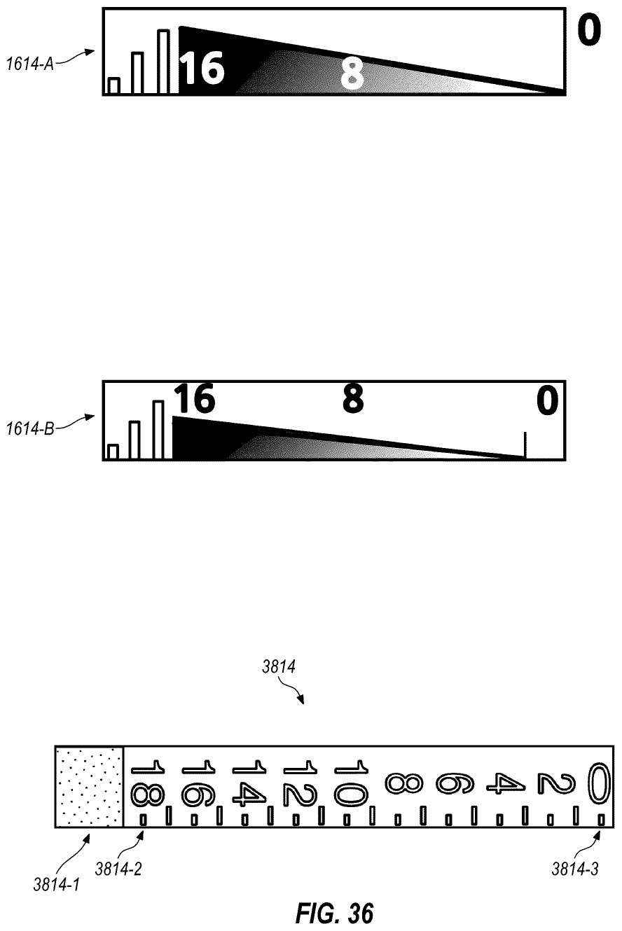

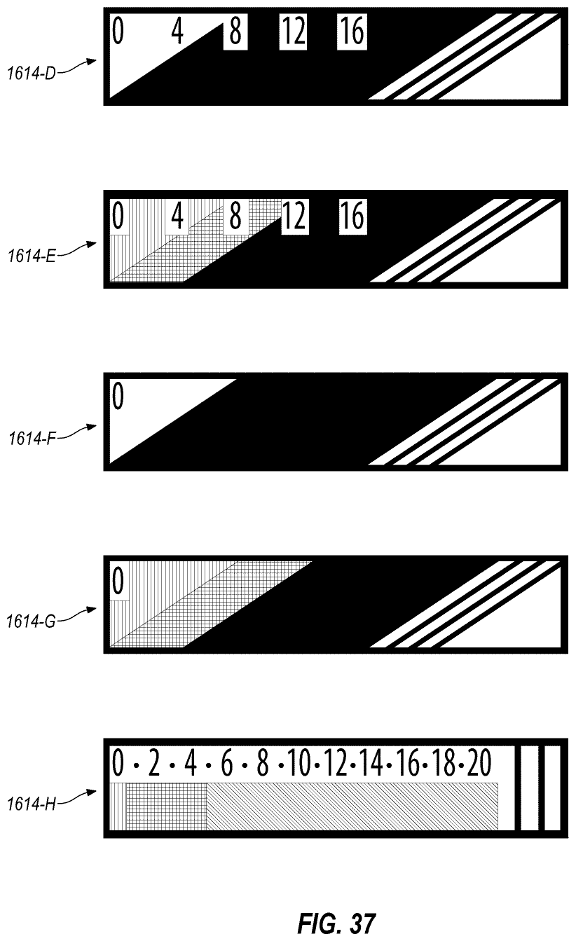

FIGS. 36 and 37 depict various visible indicia 1614-A, 1614-B, 1614-C, 1614-D, 1614-E, 1614-F, 1614-G, 1614-H, 3614 that can be applied to cams of sequential injection systems to provide various visible indicators of the state of the sequential injection systems. After the last predetermined dose delivery cycle has been completed for a particular dose delivery system, a visible indicia (e.g., “0”) is visible through the indicia window and the visible indicia may have a color indicating that the last predetermined dose delivery cycle has been completed.

After the last predetermined dose delivery cycle has been completed for a particular dose delivery system, the cam lockout rib 1629 on the cam 1604 (see FIGS. 24A and 24B) interferes with the lockout receiver 1642 on an inside of the finger flange 1605 (see FIG. 26) to prevent proximal movement of the cam 1604 relative to the finger flange 1605. Preventing proximal movement of the cam 1604 prevents the cam bypass grooves 1638 from advancing distally past the stator ribs 1644 thus preventing rotation of the cam. Preventing rotation of the cam “locks out” further dose delivery cycles by the dose delivery system 1600 after a predetermined number of dose delivery cycles. The predetermined number of dose delivery cycles may be controlled by configuring the cam 1604, cam lock at rib 1629, and lockout receiver 1642.

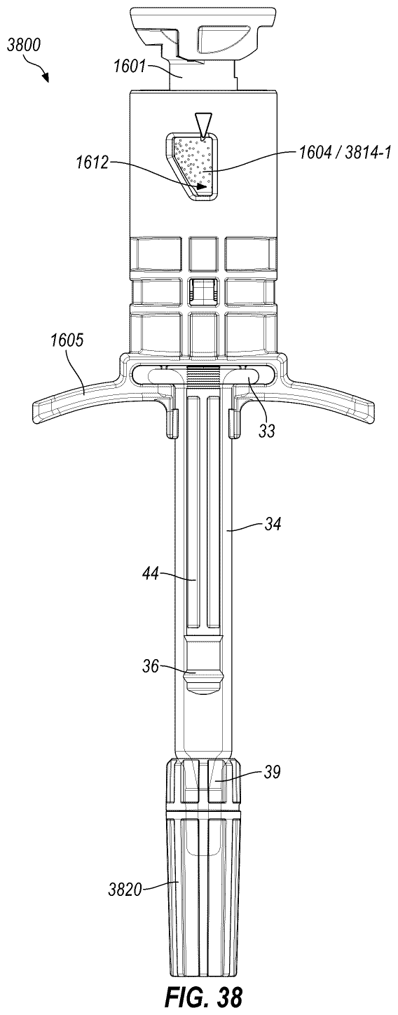

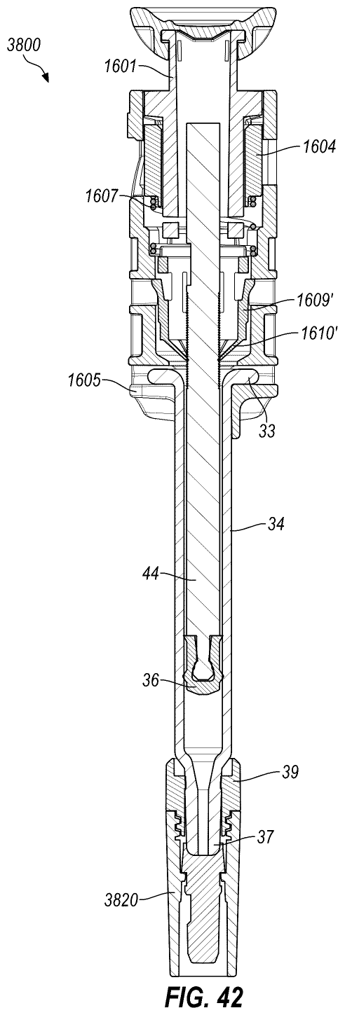

FIGS. 38 to 47 depict a serial dosing system 3800 having a single helical spring 1607 according to some embodiments. The serial dosing system 3800 depicted in FIGS. 38 to 47 is very similar to the serial dosing system 1600 depicted in FIGS. 16 to 37, and similar components have identical or similar reference numbers. The three differences between the serial dosing system 3800 depicted in FIGS. 38 to 47 and the serial dosing system 1600 depicted in FIGS. 16 to 37 are: (1) the use of the integral ratchet 1609′ depicted in FIG. 29′; (2) the presence of a system body distal connector 39; and (3) a differently designed cap 3820.

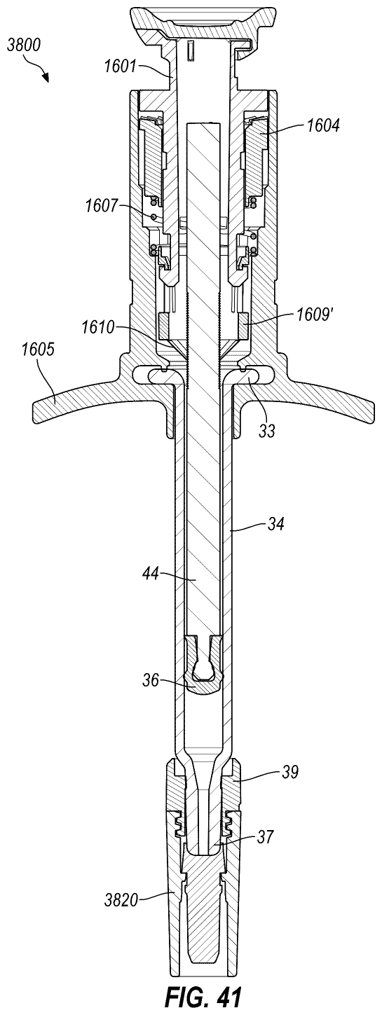

FIGS. 41 to 44 depict use of the integral ratchet 1609′ to advance the plunger member 44 using an interaction between the arms 1610′ on the integral ratchet 1609′ and the ribs 45 on the plunger member of the serial dosing system 3800.

FIGS. 38 to 42 and 46 to 47 depict the system body distal connector 39 at a distal end of the system body 34 of the serial dosing system 3800. The system body distal connector 39 may be a male threaded connector in some embodiments.

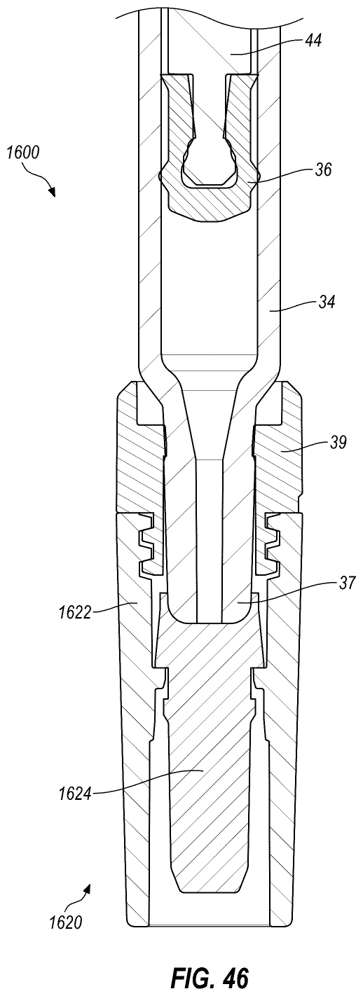

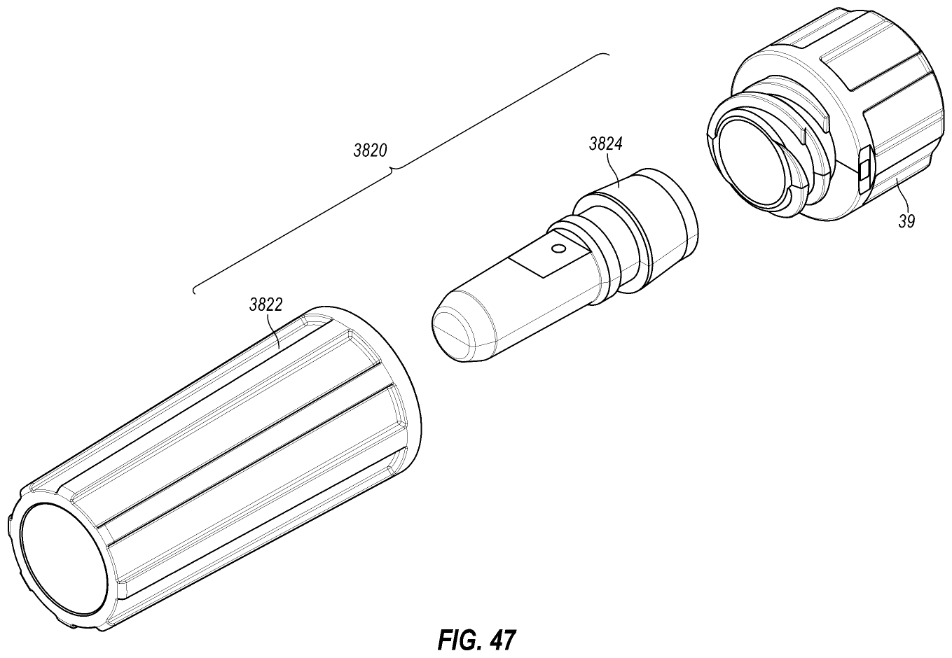

FIGS. 46 and 47 depict a cap 3820 for use with the serial dosing system 3800. The cap 3820 may have a rigid outer shell 3822 and an elastic insert 3824. The cap 3820 may also have female threads on a proximal end thereof configured to cooperate with the male threaded system body connector 39 to couple the cap 3822 the male threaded system body connector 39 and the system body 34.

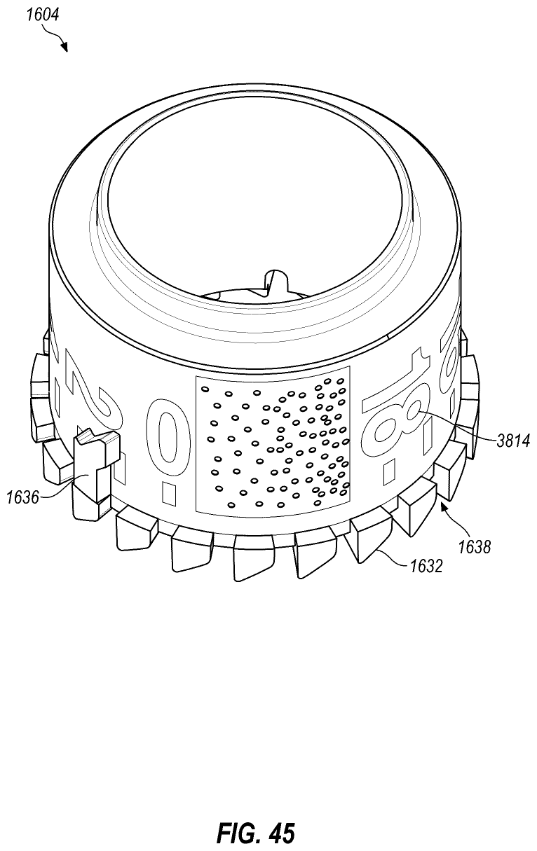

As shown in FIG. 45, the serial dosing system 3800 includes visible indicia 3814 disposed on its cam 1604. FIG. 38 depicts the serial dosing system 3800 in its storage/transport state where visible indicia 3814-1 is visible through the indicia window 1612. In the storage/transport state, the serial dosing system 3800 needs to go through one or more dose delivery cycles to expel air/dead space from the open distal end 37 of the system body 34.

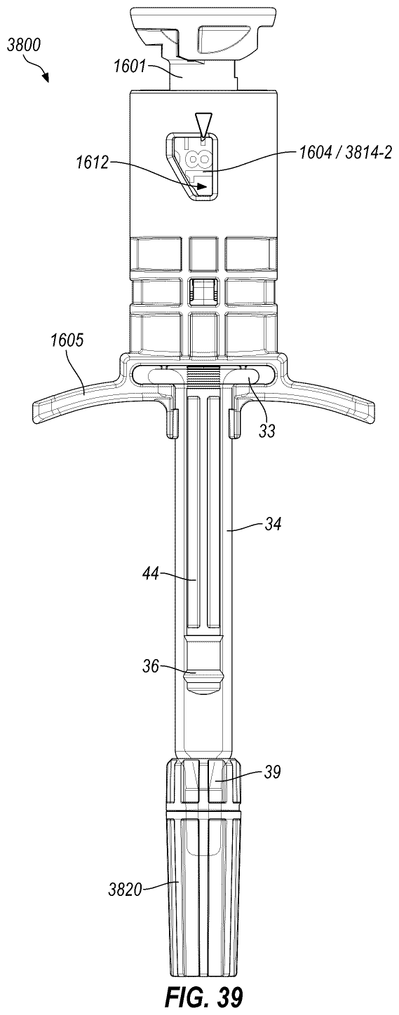

FIG. 39 depicts the serial dosing system 3800 in a ready to use state where visible indicia 3814-2 is visible through the indicia window 1612. In the ready to use state depicted in FIG. 39, a number of doses related to visible indicia 3814-2 (e.g., 18 doses) remains in the serial dosing system 3800.

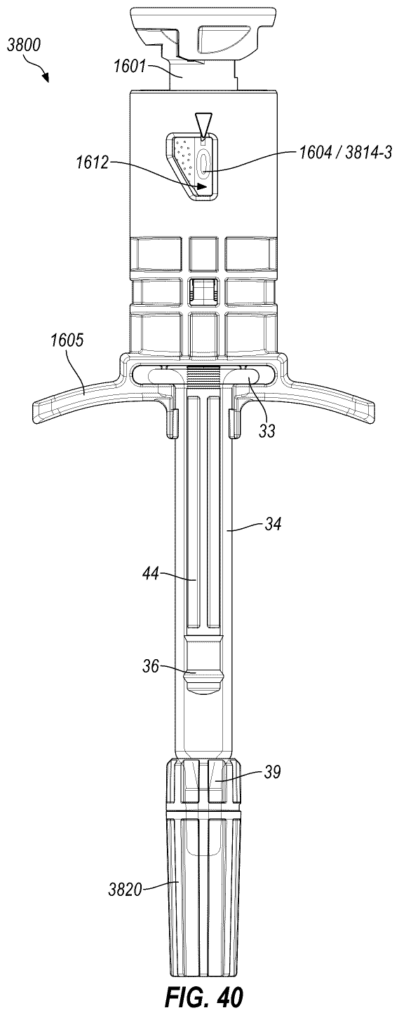

FIG. 40 depicts the serial dosing system 3800 in an all doses delivered state where visible indicia 3814-3 is visible through the indicia window 1612. In the all doses delivered state depicted in FIG. 40, a number of doses related to visible indicia 3814-3 (e.g., 0 doses) remains in the serial dosing system 3800. While this embodiment displays visible indicia 3814 of the number of doses remaining, an alternative embodiment of the device may display the number of doses already given. Pictographic illustrations and/or bar or line graphs may be used in addition to or instead of numeric indicia of the doses remaining or given. The indicia may also indicate doses which expel air from the distal end of the system body for priming of the device for dispensing and/or a needle attached for injection. At the state, the cam lockout rib 1629 on the cam 1604 (see FIGS. 24A and 24B) interferes with the lockout receiver 1642 on an inside of the finger flange 1605 (see FIG. 26) to prevent proximal movement of the cam 1604 relative to the finger flange 1605 and “lock out” the dose delivery system 3800.

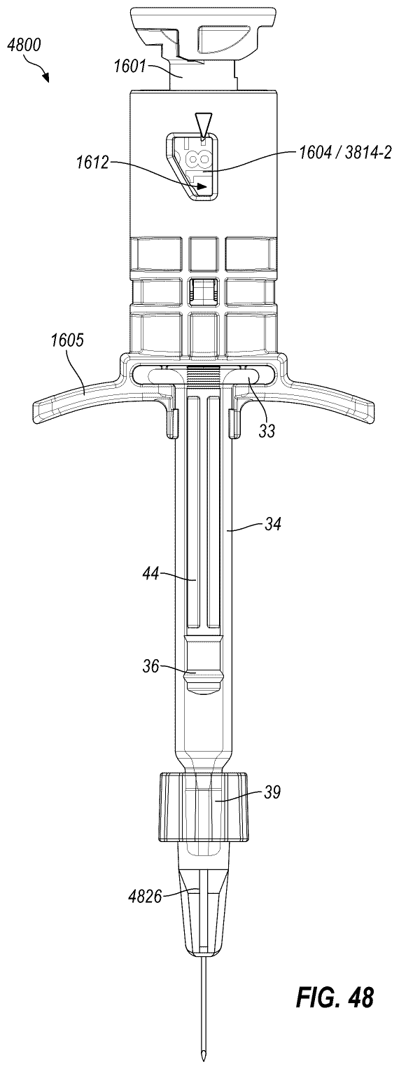

While various dose dispensing systems 600, 1600, 3800 have been described herein as systems that dispense fluid or gel from an open distal end 37 of a system body 34, the various components of the dose dispensing systems 600, 1600, 3800 described herein can also be used with dose injection systems such as the dose injection system 4800 depicted in FIG. 48. The serial dosing system 4800 depicted in FIG. 48 is very similar to the serial dosing systems 1600, 3800 depicted in FIGS. 16 to 37 and FIGS. 38 to 47, and similar components have identical or similar reference numbers. The three differences between the serial dosing system 4800 depicted in FIG. 48 and the serial dosing systems 1600, 3800 depicted in FIGS. 16 to 37 and FIGS. 38 to 47 is that instead of a cap 3820, a needle 4626 is coupled to the system body connector 39, thereby transforming the dose dispensing system 3800 to a dose injection system 4800.

While the injection systems depicted and described herein include syringes with Luer connectors, the multiple site injection systems described herein can be used with staked needles, cartridges, and pen or auto injectors, etc. The multiple site injection systems described herein can also be used with safe injection systems such as those described in U.S. patent application Ser. No. 14/696,342, the contents of which have been previously incorporated by reference herein.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. A system for medication dispensing, comprising:
a syringe body having proximal and distal ends, a syringe interior, and a syringe flange at the proximal end thereof;
a drug disposed in the syringe interior;
a finger flange coupled to the syringe flange and defining a finger flange interior and a side window therethrough;
a stopper member disposed in the syringe interior;
a plunger member coupled to the stopper member and disposed at least partially within the finger flange;
a thumbpad slidably disposed in a proximal end of the finger flange and operatively coupled to the plunger member;
a cam configured to rotate a predetermined angular distance with each full advancement of the thumbpad into the finger flange; and
a ratchet configured to advance the plunger member a predetermined longitudinal distance with each full advancement of the thumbpad into the finger flange.

2. The system of claim 1, wherein the finger flange further comprises a stator operatively coupled to the thumbpad and the cam, and configured to rotate the cam with advancement of the thumbpad into the finger flange.

3. The system of claim 1, wherein the cam includes at least one rotation indicia visible through the window in the finger flange body, and configured to indicate a number of full advancements of the thumbpad into the finger flange.

4. The system of claim 1, further comprising a spring configured to return the thumbpad proximally after distal depression of the thumbpad into the finger flange.

5. The system of claim 1, wherein the finger flange is configured to emit an audible indicator for each full depression of the thumbpad into the finger flange.

6. The system of claim 1, wherein the finger flange is configured to prevent depression of the thumbpad into the finger flange after a predetermined number of full depressions of the thumbpad into the finger flange.

7. The system of claim 1, further comprising a semi-rigid distal cap comprising:
a shield body; and
an undulating elastic member disposed in the shield body.

8. The system of claim 1, further comprising a needle coupled to a distal end of the syringe body.
